(19) United States
(12) Patent Application Publication
(10) Pub. No.: US 2023/0167442 A1
(43) Pub. Date: Jun. 1, 2023

(54) HYDROGEL-MATRIX ENCAPSULATED OLIGONUCLEOTIDES AND METHODS FOR FORMULATING AND USING ENCAPSULATED OLIGONUCLEOTIDES

(71) Applicant: BIONAUT LABS LTD., Herzliya (IL)

(72) Inventors: Tobin BROWN, Boulder, CO (US); Suehyun CHO, Torrance, CA (US); Marty STANTON, Boulder, CO (US); Alex KISELYOV, San Diego, CA (US)

(73) Assignee: BIONAUT LABS LTD., Herzliya (IL)

(21) Appl. No.: 17/894,509

(22) Filed: Aug. 24, 2022

Related U.S. Application Data
(60) Provisional application No. 63/240,809, filed on Sep. 3, 2021.
(60) Provisional application No. 63/255,394, filed on Oct. 13, 2021.

(51) Int. Cl. C12N 15/113; A61K 9/06; A61K 47/10; A61K 9/14

(52) U.S. Cl. CPC C12N 15/113; A61K 9/06; A61K 47/10; A61K 9/146; C12N 2310/11; C12N 2320/32

(57) ABSTRACT

The present invention relates to compositions of a dried rapid-release high concentration oligonucleotide-loaded PEG hydrogel-based matrix, methods for formulating hydrogel matrix-encapsulated oligonucleotides in an amount that exceeds the oligonucleotides intrinsic solubility in water or aqueous media, the hydrogel matrix-encapsulated oligonucleotides produced by the described methods, and therapeutic methods for using the dried rapid-release high concentration hydrogel matrix-encapsulated oligonucleotides for systemic and local micro-delivery.

### CROSS-REFERENCE TO RELATED APPLICATIONS

This application claims priority to U.S. Provisional Application No. 63/240,809, filed Sep. 3, 2021 and U.S. Provisional Application No. 63/255,394, filed Oct. 13, 2021, the contents of each of which are hereby incorporated by reference in their entireties.

### FIELD OF THE INVENTION

The present invention relates to dried rapid-release high concentration oligonucleotide-loaded polyethylene glycol (PEG) hydrogel-based matrices and to PEG hydrogel-based matrix-encapsulated oligonucleotides. The invention also relates to a dried rapid-release high concentration oligonucleotide-loaded thiol-maleimide PEG hydrogel-based matrix having a time required for a quantity to release half (t_{1/2}) of the oligonucleotides of from about 1 minute to about less than 30 minutes upon rehydration. The dried rapid-releases high concentration oligonucleotide-loaded thiol-maleimide PEG hydrogel-based matrix comprise greater than 40% w/w to 80% w/w or greater than 80% w/w oligonucleotide in the dried hydrogel matrix. The invention also relates to a delivery device for delivery loaded with the dried rapid-release high concentration oligonucleotide-loaded PEG hydrogel-based matrix for rapid delivery of a therapeutically effective amount of the high concentration of oligonucleotides to a specific tissue location in a subject. The invention further also relates to methods for formulating dried hydrogel matrix-encapsulated oligonucleotides in a high concentration that exceeds the oligonucleotides intrinsic solubility in water, aqueous media or body fluids. The invention also relates to the hydrogel matrix-encapsulated oligonucleotides produced by the provided methods. The invention further relates to methods for systemic and local micro-delivery of dried rapid-release high concentration therapeutic hydrogel matrix-encapsulated oligonucleotides.

### BACKGROUND OF THE INVENTION

While there are multiple formulation techniques that allow for entrapment and convenient formulation of oligonucleotides, the maximal concentration of a therapeutic oligonucleotide molecule in a matrix is determined by the oligonucleotides' inherent solubility in the aqueous media or relevant alternatives. Multiple compartments, tissues, organs, and lumens in the body may not accommodate large volumes of these oligonucleotide containing matrices due to safety reasons. As a result, the concentration of the released therapeutic oligonucleotide molecules may not reach the desired therapeutic exposure during a treatment regimen. Accordingly, there is a need for improved methods for formulating robust, reliable and reproducible formulations of oligonucleotides encapsulated in a hydrogel-based matrix, compositions comprising the oligonucleotide formulations and methods for administering therapeutically effective amounts of the encapsulated oligonucleotide formulations.

### SUMMARY OF THE INVENTION

In one aspect, the present invention provides a dried rapid-release oligonucleotide-loaded polyethylene glycol (PEG) hydrogel-based matrix for delivery of a high concentration of oligonucleotides, the oligonucleotide-loaded PEG hydrogel-based matrix having a time required for a quantity to release half (t_{1/2}) of the oligonucleotides of from about 1 minute to about less than 30 minutes upon rehydration. In some embodiments, the dried rapid-release oligonucleotide-loaded PEG hydrogel-based matrix may be optimized for carrying a large oligonucleotide (e.g., at least 1,000 bp length).

In another aspect, the present invention provides a dried rapid-release oligonucleotide-loaded thiol-maleimide PEG hydrogel-based matrix for delivery of a high concentration of oligonucleotides, the oligonucleotide-loaded thiol-maleimide PEG hydrogel-based matrix having a time required for a quantity to release half (t_{1/2}) of the oligonucleotides of from about 1 minute to about less than 30 minutes upon rehydration.

In another aspect, the present invention provides a delivery device for delivery of a therapeutically effective amount of a high concentration of oligonucleotides to a specific tissue location in a subject, wherein the delivery device is loaded with a dried rapid-release oligonucleotide-loaded PEG hydrogel-based matrix (e.g., a thiol-maleimide PEG hydrogel-based matrix), the high concentration oligonucleotide-loaded PEG hydrogel-based matrix having a time required for a quantity to release half (t_{1/2}) of the oligonucleotides during a rehydration period of from about 1 minute to less than 30 minutes.

In one aspect, the present invention provides a method for formulating dried hydrogel matrix-encapsulated oligonucleotides in a high concentration that exceeds the oligonucleotides intrinsic solubility in water, aqueous media or body fluids, the method comprising (a) reacting a mixture of a maleimide functionalized polyethylene glycol (PEG-MAL) and a polyethylene glycol compound containing sulfhydryl groups (PEG-SH) with a concentrated aqueous solution of oligonucleotides in a buffer having pH 4.0-4.8 to form an oligonucleotide-loaded hydrogel comprising a loading value of oligonucleotides of 500 to 900 μg per 1.6 μL total volume of the thiol-maleimide PEG hydrogel; (b) casting the oligonucleotide-loaded hydrogel into a mold to create a uniform oligonucleotide-loaded thiol-maleimide PEG hydrogel-based matrix; and (c) drying the uniform oligonucleotide-loaded hydrogel-based matrix at ambient temperature to form a dried oligonucleotide-loaded thiol-maleimide PEG hydrogel-based matrix comprising from greater than 40% w/w to 80% w/w or greater than 80% w/w oligonucleotide in the dried thiol-maleimide PEG hydrogel-based matrix.

In another aspect, the present invention provides a method for systemic or local micro-delivery of therapeutic oligonucleotides, the method comprising administering to a subject in need thereof 100 micron-flakes of a sliced and flattened dried rapid-release high concentration oligonucleotide-loaded PEG hydrogel-based matrix (e.g., a thiol-maleimide PEG hydrogel-based matrix), the oligonucleotide-loaded PEG hydrogel-based matrix having a time required for a quantity to release half (t_{1/2}) of the oligonucleotides of from about 1 minute to about less than 30 minutes upon rehydration.

In one aspect, the present invention provides a method for formulating hydrogel matrix-encapsulated oligonucleotides in an amount that exceeds the oligonucleotides intrinsic solubility in water or aqueous media, the method comprising: (a) reacting a mixture of a maleimide functionalized polyethylene glycol (PEG-MAL) and a polyethylene glycol compound containing sulfhydryl groups (PEG-SH) together with an aqueous solution of oligonucleotides in a buffer having pH 4.0-4.8 to form a high concentration oligonucleotide-loaded hydrogel comprising a loading value of oligonucleotides of 400 μg per 1.6 μL total volume of the thiol-maleimide PEG hydrogel; and (b) casting the high concentration oligonucleotide-loaded hydrogel into a mold to create a uniform high concentration oligonucleotide-loaded thiol-maleimide PEG hydrogel-based matrix.

In another aspect, the present invention provides a formulation of a dried hydrogel matrix-encapsulated high concentration oligonucleotides comprising a reaction mixture of a maleimide functionalized polyethylene glycol (PEG-MAL) and a polyethylene glycol compound containing sulfhydryl groups (PEG-SH) together with an aqueous solution of oligonucleotides in a buffer having pH 4.0-4.8, wherein the high concentration oligonucleotides comprise a loading value of oligonucleotides of 400 μg per 1.6 μL total volume of the thiol-maleimide PEG hydrogel.

Other features and advantages of the present invention will become apparent from the following detailed description, examples and figures. It should be understood, however, that the detailed description and the specific examples while indicating embodiments of the invention are given by way of illustration only, since various changes and modifications within the spirit and scope of the invention will become apparent to those skilled in the art from this detailed description.

### BRIEF DESCRIPTION OF THE DRAWINGS

FIGS. 1A-1C show Click chemistry for hydrogel formation and that both reactions are orthogonal to DNA functional groups. FIGS. 1A-1B show representative chemistries tested: strain promoted azide alkyne cycloaddition (SPAAC) (FIG. 1A) and Thiol-maleimide (Thiol Michael addition) (FIG. 1B). The reaction is dependent on the thiolate anion; k˜10⁶M⁻¹ s⁻¹. Both reactions orthogonal to DNA functional groups (FIG. 1C).

FIGS. 2A-2C show that thiol-Michael hydrogels have a pH-dependent reaction rate. FIG. 2A shows gel time versus pH. The buffer: was 0.1 M Histidine HCl (at various pH). Gel point measured as the time at which pipetting becomes impractical. FIG. 2B shows that at pH 4.7, a gel forms in 28 seconds. This is enough time to mix components thoroughly and cast the hydrogel into a mold to create a uniform network. FIG. 2C shows that at pH 7.4, a gel forms instantaneously upon mixing. The hydrogel is stuck in the pipette tip.

FIGS. 3A-3B show hydrogel miniaturization. The dimensional constraints were 1 mm diameter, 1-2 mm length, and a volume of 0.8 to 1.6 μL of the hydrogel mixture. FIG. 3C shows 1.6 μL of the hydrogel was cast in a pipette tip having a length of 2 mm and an average diameter of about 1 mm. FIGS. 3D-3E show the cast miniaturized hydrogels.

FIG. 4 shows DNA release studies by kinetic monitoring. 1.6 μL hydrogel was cast in pipette tip, allowed to set for 10 minutes, then removed and immersed in 1 mL water with end-over-end mixing at room temperature. DNA concentration in the supernatant was monitored with A₂₆₀ (absorption at 260 nm wavelength).

FIG. 5 shows oligonucleotide release kinetics. DNA was rapidly released from the hydrogel with a t_{1/2}˜1 min. Quantitative release was measured within 20 min. The released DNA mass correlates well with the loaded DNA mass. A_{260, min}=A_{260, 24 h}.

FIGS. 6A-6B show that the loaded oligonucleotide mass determines oligonucleotide release.

FIGS. 7A-7B show reverse phase high-performance liquid chromatography (HPLC) analysis of released DNA. FIG. 7A shows that the loaded and released DNA chromatograms are identical. The DNA that reacted with PEG is not likely to be released because the DNA is covalently attached to the hydrogel network. FIG. 7B shows solvent gradient HPLC of the hydrogel encapsulated DNA. The Column was Waters XBridge™C18 3.5 μm. Solvent A was 0.1 M triethylammonium acetate (TEAA) in water; Solvent B was 0.1 M TEAA in 80/20% (H₂O/Acetonitrile). The solvent gradient HPLC was performed at a temperature of 60° C. and a flow rate of 1 mL/min.

FIG. 8 shows hydrogel loading and release of highly concentrated DNA. The hydrogel formulation 10 μL precursor solution contained 7 μL concentrated DNA in pH 4.0 buffer, 2 μL PEG-MAL solution and 1 μL PEG-SH solution. 1.6 μL hydrogels formed, as before. 0.52 ng DNA was loaded into the hydrogel mixture and released 78% of the DNA.

FIG. 9 shows preparation of a dry hydrogel to increase loaded oligonucleotide mass. Previously made hydrogels contained 85% water by mass (6 wt % PEG and 9 wt % DNA). FIG. 9 shows that a hydrogel can therefore be made 6.7 times larger (10.7 μL) and dried to yield a DNA-loaded polymer network of the same mass (and a slightly smaller volume). DNA density was 1.4-1.7 g cm⁻³, PEG density was 1.1 g cm⁻³.

FIGS. 10A-10B show that dried hydrogels can be loaded with substantially more DNA and release is delayed during a rehydration period. A delayed, nearly linear release phase during hydrogel re-hydration (˜10 minutes by eye) was demonstrated. The t_{1/2} was 6 minutes, compared to 1 minute for hydrated hydrogels. The 905 mg DNA loaded showed a 105% release.

FIGS. 11A-11B show dried and cut oligonucleotide-hydrogel flakes and their release kinetics. The dried oligonucleotide-hydrogel of FIG. 11A was flattened and cut into small flakes. A large surface area leads to rapid burst release of DNA cargo. FIG. 11B shows that a large surface area leads to rapid burst release of the DNA cargo from the hydrogel matrix (FIG. 11B).

FIGS. 12A-12B show a comparison of DNA release rates. “Traditional” solubility-limiting immobilization of oligonucleotides (black dots “hydrogel”) and the presently described approach in Example 4 (checkered dots: “dried” hydrogel, grey dots: dried/cut hydrogel) are shown.

FIG. 13 shows eGFP plasmid transfection rate in HEK293 cells for different transfection protocols, as described in Example 5.

FIG. 14 shows the rate of eGFP plasmid DNA released in μg from days 0 through 7, based on the protocol described in Example 5.

FIG. 15 shows the μg of eGFP plasmid DNA released from two optimized hydrogel formulations after 2 days, according to the protocol described in Example 5.

FIG. 16 shows a summary of monomer gels and polymerization conditions tested as described in Example 5.

### DETAILED DESCRIPTION OF THE INVENTION

In the following detailed description, numerous specific details are set forth in order to provide a thorough understanding of the invention. However, it will be understood by those skilled in the art that this disclosure is not limited to the specific products, methods, conditions or parameters described and/or shown herein, and that the terminology used herein is for the purpose of describing particular embodiments by way of example only and is not intended to be limiting of the claimed disclosure. In other instances, well-known methods, procedures, and components have not been described in detail so as not to obscure the present invention.

The maximal concentration of a therapeutic oligonucleotide molecule in a hydrogel matrix is determined by its inherent solubility in water, aqueous media or relevant alternatives. The size of macroscopic hydrogels is usually on the order of millimeters to centimeters. Such hydrogels either are implanted surgically into the body or are placed in contact with the body for transepithelial drug delivery. Many compartments, tissues, organs, lumens in the body may not accommodate large volumes of these oligonucleotide containing-matrices due to safety reasons. As a result, the concentration of the released therapeutic oligonucleotide molecules from an administered oligonucleotide containing-matrix may not reach the desired therapeutic exposure during a treatment regimen.

To resolve the aforementioned limitations of conventional oligonucleotide containing-matrices, the present invention provides a methodology for robust, reliable and reproducible formulation of oligonucleotides in polyethylene glycol (PEG) hydrogels. The herein provided approach uses hydrogel-based matrix to encapsulate diverse oligonucleotides in an amount that dramatically exceeds the oligonucleotides' intrinsic solubility in water or aqueous media. The provided methods rely on preparation of stable, well-characterized super-concentrated, i.e., having a high concentration of oligonucleotides comprising greater than 40% w/w to 80% w/w or greater than 80% w/w oligonucleotide in the dried hydrogel matrix, dried hydrogel solution(s), dried and processed hydrogels and/or colloidal systems containing an oligonucleotide of interest that is entrapped by in situ forming hydrogels. The resulting formulation of a dried rapid-release high concentration oligonucleotide-loaded PEG hydrogel-based matrix (e.g., a thiol-maleimide PEG hydrogel-based matrix) is suitable for both systemic and local (micro)delivery of therapeutic oligonucleotides, especially to the anatomical loci that are particularly sensitive to external interventions, as exemplified by the CNS, including the brain and the spine.

Unless otherwise defined herein, scientific and technical terms used in connection with this disclosure shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In this disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the term “nucleic acid” refers to polynucleotides or to oligonucleotides such as deoxyribonucleic acid (DNA), and, where appropriate, ribonucleic acid (RNA) or mimetics thereof. This term should also be understood to include, as equivalents, analogs of either RNA or DNA made from nucleotide analogs, and, as applicable to the embodiment being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides may be used in place of native forms of oligonucleotides because of desirable properties such as, for example, enhanced cellular uptake, enhanced affinity for nucleic acid target and increased stability in the presence of nucleases.

Throughout this disclosure, various embodiments may be presented in a range format. It should be understood that a description in range format is merely for convenience and brevity and should not be construed as an inflexible limitation on the scope of the invention. Accordingly, description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals therebetween.

When values are expressed as approximations, by use of the antecedent “about,” it is understood that the particular value forms another embodiment. All ranges are inclusive and combinable. In one embodiment, the term “about” refers to a deviance of between 0.1-5% from the indicated number or range of numbers.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, when referring to a measurable value such as an amount, a temporal duration, a concentration, and the like, may encompass variations of ±20% or ±10%, more specifically ±5%, even more particularly ±1%, and still more preferably ±0.1% from the specified value, as such variations are appropriate to perform the disclosed methods. In another embodiment, the term “about” refers to a deviance of between 1-10% from the indicated number or range of numbers. In another embodiment, the term “about” refers to a deviance of up to 20% from the indicated number or range of numbers. In one embodiment, the term “about” refers to a deviance of ±10% from the indicated number or range of numbers. In another embodiment, the term “about” refers to a deviance of ±5% from the indicated number or range of numbers.

The terms “subject,” “individual,” and “patient” are used interchangeably herein, and refer to an animal, for example a human, including a human in need of therapy for, or susceptible to, a condition or its sequelae. to whom treatment, including prophylactic treatment, with the pharmaceutical composition according to the present invention, is provided. The term “subject” does not exclude an individual that is normal in all respects. The term “subject” as used herein refers to human and non-human animals. The terms “non-human animals” and “non-human mammals” are used interchangeably herein and include all vertebrates, e.g., mammals, such as non-human primates (particularly higher primates), sheep, dog, rodent, (e.g., mouse or rat), guinea pig, goat, pig, cat, rabbits, cows, horses and non-mammals such as reptiles, amphibians, chickens, and turkeys.

As used herein, the terms “component,” “composition,” “composition of compounds,” “compound,” “drug,” “pharmacologically active agent,” “active agent,” “active ingredient,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

Thus, as used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In an embodiment, pharmaceutical compositions containing the therapeutic agent or agents described herein, can be, in one embodiment, administered to a subject by any method known to a person skilled in the art, such as, without limitation, orally, parenterally, transnasally, transmucosally, subcutaneously, transdermally, intramuscularly, intravenously, intraarterially, intra-dermally, intra-peritoneally, intra-ventricularly, intra-cranially, intra-vaginally, or intra-tumorally.

Carriers may be any of those conventionally used, as described above, and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. In one embodiment, water, preferably bacteriostatic water, is the carrier when the pharmaceutical composition is administered intravenously or intratumorally. Saline solutions and aqueous dextrose and glycerol solutions can also be employed as liquid carriers, particularly for injectable solutions.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include, without limitation, physiological saline, bacteriostatic water, Cremophor EL.TM. (BASF, Parsippany, N.J.) or phosphate buffered saline (PBS). The composition should be sterile and should be fluid to the extent that easy syringeability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as appropriate, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The compositions and formulations as described herein may be administered alone or with other biologically-active agents. Administration can be systemic or local, e.g. through portal vein delivery to the liver. In addition, it may be advantageous to administer the composition into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the therapeutic oligonucleotide locally to the area in need of treatment; this may be achieved by, for example, and not by way of limitation, local infusion during surgery, topical application, by injection, by means of a catheter, by means of a suppository, or by means of an implant.

Moreover, “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable” also includes those carriers approved by a regulatory agency of the Federal or a state government or listed in the U.S. Pharmacopeia or other generally recognized pharmacopeia for use in animals and, more particularly, in humans.

In one aspect, the present invention provides a dried rapid-release oligonucleotide-loaded thiol-maleimide PEG hydrogel-based matrix for delivery of a high concentration of oligonucleotides, the oligonucleotide-loaded thiol-maleimide PEG hydrogel-based matrix having a time required for a quantity to release half (t_{1/2}) of the oligonucleotides of from about 1 minute to about less than 30 minutes upon rehydration. In an embodiment of the provided dried rapid-release oligonucleotide-loaded thiol-maleimide PEG hydrogel-based matrix, the high concentration of oligonucleotides comprises greater than 40% w/w to 80% w/w or greater than 80% w/w oligonucleotide in the dried hydrogel. In some embodiments, the high concentration of oligonucleotides comprises greater than 40% w/w to 90% w/w or greater than 90% w/w oligonucleotide in the dried hydrogel. In various embodiments, the high concentration of oligonucleotides comprises greater than 40% w/w to 95% w/w or greater than 95% w/w oligonucleotide in the dried hydrogel. In some embodiments, the high concentration of oligonucleotides comprises greater than 50% w/w oligonucleotide in the dried hydrogel. In certain embodiments, the high concentration of oligonucleotides comprises 60% w/w or greater than 60% w/w oligonucleotide in the dried hydrogel. In an embodiment, the high concentration of oligonucleotides comprises 60% w/w or greater than 60% w/w oligonucleotide in the dried hydrogel. In some embodiments, the high concentration of oligonucleotides comprises 70% w/w or greater than 70% w/w oligonucleotide in the dried hydrogel. In various embodiments, the high concentration of oligonucleotides comprises 80% w/w or greater than 80% w/w oligonucleotide in the dried hydrogel. In a particular embodiment of the dried rapid-release oligonucleotide-loaded thiol-maleimide PEG hydrogel-based matrix, the high concentration of oligonucleotides comprises greater than 80% w/w oligonucleotide in the dried hydrogel. In some embodiments of the dried rapid-release oligonucleotide-loaded thiol-maleimide PEG hydrogel-based matrix, the high concentration of oligonucleotides comprises 90% w/w or greater than 90% w/w oligonucleotide in the dried hydrogel. In an embodiment of the dried rapid-release oligonucleotide-loaded thiol-maleimide PEG hydrogel-based matrix, the high concentration of oligonucleotides comprises 95% w/w oligonucleotide in the dried hydrogel. In some embodiments, the high concentration of oligonucleotides comprises greater than 95% w/w oligonucleotide in the dried hydrogel.

In an embodiment, the dried rapid-release oligonucleotide-loaded thiol-maleimide PEG hydrogel-based matrix has a t_{1/2} of the oligonucleotides of from about 1 minute to about less than 20 minutes upon rehydration in water, aqueous media or body fluids selected from the group consisting of cerebrospinal fluid (CSF), blood, lymph, synovial fluid or aqueous humor. In a particular embodiment, the dried rapid-release oligonucleotide-loaded thiol-maleimide PEG hydrogel-based matrix has a t_{1/2} of the oligonucleotides of from about 1 minute to about less than 10 minutes upon rehydration. In some embodiments, the dried rapid-release oligonucleotide-loaded thiol-maleimide PEG hydrogel-based matrix has a t_{1/2} of the oligonucleotides of from about 1 minute to about less than 6 minutes upon rehydration. In certain embodiments, the dried rapid-release oligonucleotide-loaded thiol-maleimide PEG hydrogel-based matrix has a t_{1/2} of the oligonucleotides of about 1 minute upon rehydration.

In another aspect, the present invention provides a dried rapid-release oligonucleotide-loaded PEG hydrogel-based matrix for delivery of a high concentration of oligonucleotides, the oligonucleotide-loaded PEG hydrogel-based matrix having a time required for a quantity to release half (t_{1/2}) of the oligonucleotides of from about 1 minute to about less than 30 minutes upon rehydration. In some such embodiments, the PEG hydrogel-based matrix is a polyethylene glycol—polylactic acid—diacrylate (PEG-PLA-DA) hydrogel. In some such embodiments, the PEG-PLA-DA hydrogel is photo-polymerized. In some such embodiments, the PEG hydrogel-based matrix is a PEG-diacrylate (PEG-DA) hydrogel. In some such embodiments, the PEG-DA hydrogel is photo-polymerized. In some such embodiments, the PEG hydrogel-based matrix comprises methoxy-PEG-acrylate (mPEG-A). In some such embodiments, the mPEG-A is photo-polymerized. In some such embodiments, the PEG hydrogel-based matrix is a hydrogel formed with thiol-Michael Click chemistry, as described throughout the present disclosure.

In an embodiment of the provided dried rapid-release oligonucleotide-loaded PEG hydrogel-based matrix, the high concentration of oligonucleotides comprises greater than 40% w/w to 80% w/w or greater than 80% w/w oligonucleotide in the dried hydrogel. In some embodiments, the high concentration of oligonucleotides comprises greater than 40% w/w to 90% w/w or greater than 90% w/w oligonucleotide in the dried hydrogel. In various embodiments, the high concentration of oligonucleotides comprises greater than 40% w/w to 95% w/w or greater than 95% w/w oligonucleotide in the dried hydrogel. In some embodiments, the high concentration of oligonucleotides comprises greater than 50% w/w oligonucleotide in the dried hydrogel. In certain embodiments, the high concentration of oligonucleotides comprises 60% w/w or greater than 60% w/w oligonucleotide in the dried hydrogel. In an embodiment, the high concentration of oligonucleotides comprises 60% w/w or greater than 60% w/w oligonucleotide in the dried hydrogel. In some embodiments, the high concentration of oligonucleotides comprises 70% w/w or greater than 70% w/w oligonucleotide in the dried hydrogel. In various embodiments, the high concentration of oligonucleotides comprises 80% w/w or greater than 80% w/w oligonucleotide in the dried hydrogel. In a particular embodiment of the dried rapid-release oligonucleotide-loaded PEG hydrogel-based matrix, the high concentration of oligonucleotides comprises greater than 80% w/w oligonucleotide in the dried hydrogel. In some embodiments of the dried rapid-release oligonucleotide-loaded PEG hydrogel-based matrix, the high concentration of oligonucleotides comprises 90% w/w or greater than 90% w/w oligonucleotide in the dried hydrogel. In an embodiment of the dried rapid-release oligonucleotide-loaded PEG hydrogel-based matrix, the high concentration of oligonucleotides comprises 95% w/w oligonucleotide in the dried hydrogel. In some embodiments, the high concentration of oligonucleotides comprises greater than 95% w/w oligonucleotide in the dried hydrogel.

In an embodiment, the dried rapid-release oligonucleotide-loaded PEG hydrogel-based matrix has a t_{1/2} of the oligonucleotides of from about 1 minute to about less than 20 minutes upon rehydration in water, aqueous media or body fluids selected from the group consisting of cerebrospinal fluid (CSF), blood, lymph, synovial fluid or aqueous humor. In a particular embodiment, the dried rapid-release oligonucleotide-loaded PEG hydrogel-based matrix has a t_{1/2} of the oligonucleotides of from about 1 minute to about less than 10 minutes upon rehydration. In some embodiments, the dried rapid-release oligonucleotide-loaded PEG hydrogel-based matrix has a t_{1/2} of the oligonucleotides of from about 1 minute to about less than 6 minutes upon rehydration. In certain embodiments, the dried rapid-release oligonucleotide-loaded PEG hydrogel-based matrix has a t_{1/2} of the oligonucleotides of about 1 minute upon rehydration.

In some embodiments, the dried rapid-release oligonucleotide-loaded PEG hydrogel-based matrix may be optimized for carrying a large oligonucleotide (e.g., at least 1,000 bp length). In such embodiments, the large oligonucleotide may be at least 1,000 bp, at least 1.5 kbp, at least 2.0 kbp, at least 2.5 kbp, at least 3.0 kbp, at least 3.5 kbp, at least 4.0 kbp, at least 4.5 kbp, at least 5.0 kbp, at least 5.4 kbp, at least 5.5 kbp, at least 6.0 kbp in length, or larger. The large oligonucleotide may be linear or circular. The large oligonucleotide may be an expression vector, such as a plasmid. Such embodiments may be further understood with reference to Example 5 and Table 2. In some such embodiments, the hydrogel is a polyethylene glycol—polylactic acid—diacrylate (PEG-PLA-DA) hydrogel. In some such embodiments, the PEG-PLA-DA hydrogel is photo-polymerized, e.g., with 385 nm light, about 25 mW cm⁻² flux for about 5 minutes, with 0.2% photoinitiator. In some such embodiments, the hydrogel is a PEG-diacrylate (PEG-DA) hydrogel. The PEG-DA hydrogel is photo-polymerized, e.g., with 385 nm light, about 25 mW cm⁻² flux for about 5 minutes, with 0.2% photoinitiator. In some such embodiments, the PEG hydrogel-based matrix comprises methoxy-PEG-acrylate (mPEG-A). In some such embodiments, the mPEG-A is photo-polymerized, e.g., with 385 nm light, about 25 mW cm⁻² flux for about 5 minutes, with 0.2% photoinitiator. In some such embodiments, the hydrogel is is formed with thiol-Michael Click chemistry, as described throughout the present disclosure, e.g., the thiol-Michael hydrogel is polymerized by an about-10-minute exposure to a solution of pH of about 4.0. In some such embodiments, the oligonucleotide-loaded PEG hydrogel-based matrix comprises sucrose. In some such embodiments, the sucrose to DNA ratio (by mass) ranges from about 160:1 to about 500:1.

In various embodiments, the dried rapid-release oligonucleotide-loaded PEG hydrogel-based matrix (e.g., a thiol-maleimide PEG hydrogel-based matrix) has an average length of 20 μm. In some embodiments, the dried rapid-release oligonucleotide-loaded PEG hydrogel-based matrix has an average length of 15 atm. In certain embodiments, the dried rapid-release oligonucleotide-loaded PEG hydrogel-based matrix has an average length of 10 μm. In a particular embodiment, the dried rapid-release oligonucleotide-loaded PEG hydrogel-based matrix has an average length of between 1 μm and 10 μm. In an embodiment, the dried rapid-release oligonucleotide-loaded PEG hydrogel-based matrix has an average length of between 1 μm and 5 μm. In certain embodiments, the dried rapid-release oligonucleotide-loaded PEG hydrogel-based matrix has an average length of between 1 μm and 2 μm. In a particular embodiment, the dried rapid-release oligonucleotide-loaded PEG hydrogel-based matrix has an average length of 1 μm.

In some embodiments of the dried rapid-release oligonucleotide-loaded PEG hydrogel-based matrix (e.g., a thiol-maleimide PEG hydrogel-based matrix), the oligonucleotides comprise 25-mer poly-dT(s). In various embodiments of the dried rapid-release oligonucleotide-loaded PEG hydrogel-based matrix, wherein density of the oligonucleotides is 1.4-1.7 g cm⁻³ and PEG density is 1.1 g⁻³ in a volume of 10.6 μL of the dried rapid-release oligonucleotide-loaded PEG hydrogel-based matrix. In certain embodiments, the dried rapid-release oligonucleotide-loaded PEG hydrogel-based matrix releases from 0.025 mg to 1 mg of the oligonucleotides per 1.6 μL total volume of the PEG hydrogel during a rehydration period of from less than one minute to 15 minutes. In some embodiments, the dried rapid-release high concentration oligonucleotide-loaded PEG hydrogel-based matrix comprises sliced and flattened flakes of between 1 micron and 100 microns in thickness. As used herein, the “100 micron-flakes” are 100 microns in thickness. In an embodiment of the dried rapid-release oligonucleotide-loaded PEG hydrogel-based matrix, the sliced and flattened 1 micron- to 100 micron-flakes release 0.1 mg to 0.4 mg of about 1 mg of loaded oligonucleotide mass in a near instantaneous-release during a rehydration period of from less than one minute to about less than 10 minutes. In some embodiments of the dried rapid-release oligonucleotide-loaded PEG hydrogel-based matrix, the sliced and flattened 100 (or smaller thickness) micron-flakes release >95% of the oligonucleotides of about 1 mg loaded oligonucleotide mass during a rehydration period of from about 15 to about 35 minutes. In an embodiment, the dried rapid-release high concentration oligonucleotide-loaded PEG hydrogel-based matrix of having a 1 mm to 2 mm thickness is cut/sliced and flattened further to ˜100 micron or smaller thickness. In various embodiments of the dried rapid-release oligonucleotide-loaded PEG hydrogel-based matrix, the sliced and flattened 100 micron (or smaller) flakes release 100% of the oligonucleotides of about 1 mg loaded oligonucleotide mass during a rehydration period of from about 15 to about 35 minutes. In some embodiments, the dried rapid-release oligonucleotide-loaded PEG hydrogel-based matrix is loadable into a delivery device having a volume of 1 mm length by 1 mm width by 1 mm height.

In an embodiment, the dried rapid-release oligonucleotide-loaded thiol-maleimide PEG hydrogel-based matrix comprises a reaction mixture of a maleimide functionalized polyethylene glycol (PEG-MAL) and a polyethylene glycol compound containing sulfhydryl groups (PEG-SH) together with an aqueous solution of oligonucleotides in a buffer having pH 4.0-4.8, wherein the oligonucleotides comprise a loading value of oligonucleotides of 500 to 900 micrograms per 1.6 μL total volume of the thiol-maleimide PEG hydrogel. In some embodiments, the dried rapid-release oligonucleotide-loaded thiol-maleimide PEG hydrogel-based matrix is cast in a mold.

In particular embodiments, the mold-cast dried rapid-release oligonucleotide-loaded thiol-maleimide PEG hydrogel-based matrix has length and diameter dimensions of from a micron scale to a 2 mm length×1 to 2 mm diameter. In certain embodiments of the mold-cast dried rapid-release oligonucleotide-loaded thiol-maleimide PEG hydrogel-based matrix, the mold is a conventional pipette tip and the dried oligonucleotide-loaded thiol-maleimide PEG hydrogel-based matrix comprises a microcylinder of 2 mm length×1 mm diameter dimensions.

In another aspect, the present invention provides a delivery device for delivery of a therapeutically effective amount of a high concentration of oligonucleotides to a specific tissue location in a subject, wherein the delivery device is loaded with a dried rapid-release oligonucleotide-loaded PEG hydrogel-based matrix (e.g., a thiol-maleimide PEG hydrogel-based matrix), the high concentration oligonucleotide-loaded PEG hydrogel-based matrix having a time required for a quantity to release half (t_{1/2}) of the oligonucleotides during a rehydration period of from about 1 minute to less than 30 minutes. In an embodiment, the delivery device has a volume of 1 mm length by 1 mm width by 1 mm height. In some embodiments of the delivery device, the high concentration of oligonucleotides in the dried rapid-release oligonucleotide-loaded PEG hydrogel-based matrix comprises from greater than 40% w/w to 80% w/w or greater than 80% w/w oligonucleotide in the dried hydrogel-based matrix. In various embodiments, the high concentration of oligonucleotides in the dried rapid-release oligonucleotide-loaded PEG hydrogel-based matrix comprises greater than 50% w/w oligonucleotide in the dried hydrogel-based matrix. In an embodiment, the high concentration of oligonucleotides in the dried rapid-release oligonucleotide-loaded PEG hydrogel-based matrix comprises 60% w/w or greater than 60% w/w oligonucleotide in the dried hydrogel-based matrix. In some embodiments of the delivery device, the high concentration of oligonucleotides in the dried rapid-release oligonucleotide-loaded PEG hydrogel-based matrix comprises 70% w/w or greater than 70% w/w oligonucleotide in the dried hydrogel-based matrix. In various embodiments of the delivery device, the high concentration of oligonucleotides in the dried rapid-release oligonucleotide-loaded PEG hydrogel-based matrix comprises 80% w/w or greater than 80% w/w oligonucleotide in the dried hydrogel-based matrix. In certain embodiments of the delivery device, the high concentration of oligonucleotides in the dried rapid-release oligonucleotide-loaded PEG hydrogel-based matrix comprises 90% w/w or greater than 90% w/w oligonucleotide in the dried hydrogel-based matrix. In an embodiment of the delivery device, the high concentration of oligonucleotides in the dried rapid-release oligonucleotide-loaded PEG hydrogel-based matrix comprises 95% w/w oligonucleotide in the dried hydrogel-based matrix. In some embodiments of the delivery device, the high concentration of oligonucleotides in the dried rapid-release oligonucleotide-loaded PEG hydrogel-based matrix comprises greater than 95% w/w oligonucleotide in the dried hydrogel-based matrix.

In a particular embodiment of the delivery device, the dried rapid-release oligonucleotide-loaded PEG hydrogel-based matrix (e.g., a thiol-maleimide PEG hydrogel-based matrix) has a t_{1/2} of the oligonucleotides of from about 1 minute to less than about 20 minutes upon rehydration. In some embodiments, the dried rapid-release oligonucleotide-loaded PEG hydrogel-based matrix has a t_{1/2} of the oligonucleotides of from about 1 minute to about less than 10 minutes upon rehydration. In certain embodiments, the dried rapid-release oligonucleotide-loaded PEG hydrogel-based matrix has a t_{1/2} of the oligonucleotides of from about 1 minute to less than 6 minutes upon rehydration. In various embodiments, the dried rapid-release oligonucleotide-loaded PEG hydrogel-based matrix has a t_{1/2} of the oligonucleotides of about 1 minute upon rehydration.

In some embodiments of the delivery device, the dried rapid-release oligonucleotide-loaded PEG hydrogel-based matrix (e.g., a thiol-maleimide PEG hydrogel-based matrix) has an average length of 20 μm. In certain embodiments, the dried rapid-release oligonucleotide-loaded PEG hydrogel-based matrix has an average length of 15 μm. In various embodiments, the dried rapid-release oligonucleotide-loaded PEG hydrogel-based matrix has an average length of 10 μm. In particular embodiments, the dried rapid-release oligonucleotide-loaded PEG hydrogel-based matrix has an average length of between 1 μm and 10 μm. In some embodiments of the delivery device, the dried rapid-release oligonucleotide-loaded PEG hydrogel-based matrix has an average length of between 1 μm and 5 μm. In a particular embodiment, the dried rapid-release oligonucleotide-loaded PEG hydrogel-based matrix has an average length of between 1 μm and 2 μm. In certain embodiments, the dried rapid-release oligonucleotide-loaded PEG hydrogel-based matrix has an average length of 1 μm.

In one aspect, the present invention provides a method for formulating dried rapid-release high concentration hydrogel matrix-encapsulated oligonucleotides in a high concentration that exceeds the oligonucleotides intrinsic solubility in water, aqueous media or body fluids, the method comprising (a) reacting a mixture of a maleimide functionalized polyethylene glycol (PEG-MAL) and a polyethylene glycol compound containing sulfhydryl groups (PEG-SH) with a concentrated aqueous solution of oligonucleotides in a buffer having pH 4.0-4.8 to form an oligonucleotide-loaded hydrogel comprising a loading value of oligonucleotides of 500 to 900 μg per 1.6 μL total volume of the thiol-maleimide PEG hydrogel; (b) casting the oligonucleotide-loaded hydrogel into a mold to create a uniform oligonucleotide-loaded thiol-maleimide PEG hydrogel-based matrix; and (c) drying the uniform oligonucleotide-loaded hydrogel-based matrix at ambient temperature to form a dried oligonucleotide-loaded thiol-maleimide PEG hydrogel-based matrix comprising from greater than 40% w/w to 80% w/w or greater than 80% w/w oligonucleotide in the dried thiol-maleimide PEG hydrogel-based matrix. In some embodiments, the drying of the uniform oligonucleotide-loaded hydrogel-based matrix at ambient temperature occurs for 72 hours. In an embodiment, the method further comprises preparing the concentrated aqueous solution of oligonucleotides by heating an aqueous solution of oligonucleotides to 60° C. with simultaneous sonication. In some embodiments of the provided methods, the aqueous solution of oligonucleotides comprises 25-mer poly-dT. In particular embodiments, the method further comprises slicing the dried oligonucleotide-loaded thiol-maleimide PEG hydrogel-based matrix into 100 micron-flakes.

In some embodiments of above-described methods, density of the oligonucleotides is 1.4-1.7 g cm⁻³ and the PEG density is 1.1 g⁻³ in a volume of 10.6 μL of the dried oligonucleotide-loaded thiol-maleimide PEG hydrogel-based matrix. In certain embodiments of the methods, the dried oligonucleotide-loaded thiol-maleimide PEG hydrogel-based matrix releases from 0.025 mg to 1 mg of the oligonucleotides per 1.6 μL total volume of the thiol-maleimide PEG hydrogel during a rehydration period of about 1 minute to about 15 minutes. In particular embodiments of the methods, a time required for a quantity to release half (t_{1/2}) of the oligonucleotides from the dried oligonucleotide-loaded thiol-maleimide PEG hydrogel-based matrix in the water, aqueous media or body fluids is from about 1 minute to 6 minutes during a rehydration period. In some embodiments of the provided methods, the 100 micron-flakes of the dried oligonucleotide-loaded thiol-maleimide PEG hydrogel-based matrix release the oligonucleotides in a near instantaneous release of less than one minute during rehydration in water, aqueous media or body fluids. In an embodiment of the methods, from 550 μg to 997.50 μg of oligonucleotides of from 950 μg to 1 mg of a loaded oligonucleotide mass is released during a rehydration period of from about 15 to 35 minutes. In some embodiments, the mold has length and diameter dimensions on a micron scale up to a 2 mm length×10 mm diameter. In a particular embodiment, the mold is a conventional pipette tip having length and diameter dimensions of 2 mm length×1 to 2 mm diameter and the casting forms a microcylinder. In certain embodiments, the microcylinder releases 110 μg of the oligonucleotides within 1 minute post-immersion/rehydration in water, aqueous media or body fluids or organs.

In another aspect, the present invention provides a method for systemic or local micro-delivery of therapeutic oligonucleotides, the method comprising administering to a subject in need thereof 100 micron-flakes of a sliced and flattened dried rapid-release high concentration oligonucleotide-loaded PEG hydrogel-based matrix (e.g., a thiol-maleimide PEG hydrogel-based matrix), the oligonucleotide-loaded PEG hydrogel-based matrix having a time required for a quantity to release half (t_{1/2}) of the oligonucleotides of from about 1 minute to about less than 30 minutes upon rehydration. In some embodiments of the provided method, the 100 micron-flakes of the sliced and flattened dried rapid-release high concentration oligonucleotide-loaded PEG hydrogel-based matrix are administered to the central nervous system by implantation of the 100 micron-flakes to an anatomical locus of the subject for local micro-delivery of the therapeutic oligonucleotides. In a particular embodiment, the anatomical locus is a brain or a spine. In certain embodiments, the 100 micron-flakes are administered systemically by an enteral or parenteral administration. In various embodiments, the method further comprises preparing the 100 micron-flakes, the method comprising: (a) casting into a mold having length and diameter dimensions of from a micron scale up to a 2 mm length×1 mm diameter a high concentration oligonucleotide-loaded thiol-maleimide PEG hydrogel comprising a loading value of oligonucleotides of 500 to 900 μg per 1.6 μL total volume of the thiol-maleimide PEG hydrogel to create a uniform oligonucleotide-loaded thiol-maleimide PEG hydrogel-based matrix; (b) drying the uniform oligonucleotide-loaded hydrogel-based matrix at ambient temperature to form a dried oligonucleotide-loaded thiol-maleimide PEG hydrogel-based matrix comprising from greater than 40% w/w to 95% w/w oligonucleotide in the dried thiol-maleimide PEG hydrogel-based matrix; (c) slicing the dried thiol-maleimide PEG hydrogel-based matrix into 100 micron-flakes; and (d) flattening the flakes to between 1 micron and 100 microns in thickness. In particular embodiments, the rehydration of the sliced and flattened flakes of the dried high concentration oligonucleotide-loaded PEG hydrogel occurs at the time of or after systemic or local micro-delivery of the therapeutic oligonucleotides, as discussed herein, in less than one minute up to about to 35 minutes after administration to the anatomical locus or after enteral or parenteral administration; the rehydration takes place in the blood or the body fluids selected from the group consisting of cerebrospinal fluid (CSF), blood, lymph, synovial fluid or aqueous humor, into which the flakes are administered or delivered by the systemic or local micro-delivery to the anatomical locus, such as an organ of the subject, including but not limited to a brain or a spine.

In some embodiments of the provided methods, the high concentration oligonucleotide-loaded thiol-maleimide PEG hydrogel is cast into a conventional pipette tip to form a microcylinder of 2 mm length×1 to 2 mm diameter dimensions. In certain embodiments, the microcylinder releases 110 μg of the oligonucleotides within 1 minute post-immersion/rehydration in water, aqueous media, or body fluids or organs. In some embodiments, the drying of the uniform oligonucleotide-loaded hydrogel-based matrix at ambient temperature is for 72 hours. In various embodiments, the method further comprises preparing the high concentration oligonucleotides by heating an aqueous solution of oligonucleotides comprising 20 to 50% w/w (or greater than 50% w/w) oligonucleotide in the aaqueous solution to 60° C. with simultaneous sonication. In embodiments of the provided methods, the aqueous solution of oligonucleotides comprises 25-mer poly-dT. In some embodiments of the provided methods, the dried aqueous solution of oligonucleotides comprises 25-mer poly-dT. In a particular embodiment, the 100 micron-flakes have a density of the oligonucleotides of from 1.4 to 1.7 g cm⁻³. In certain embodiments, the dried oligonucleotide-loaded thiol-maleimide PEG hydrogel-based matrix comprises from a 60% w/w to 40% w/w ratio to a 80% w/w to 20% w/w ratio of the oligonucleotide to the thiol-maleimide PEG. In some embodiments, the dried oligonucleotide-loaded thiol-maleimide PEG hydrogel-based matrix comprises from a 60% w/w to 40% w/w ratio to a 95% w/w to 5% w/w ratio of the oligonucleotide to the thiol-maleimide PEG. In particular embodiments of the provided methods, wherein the dried oligonucleotide-loaded thiol-maleimide PEG hydrogel-based matrix releases from 0.025 mg to 1 mg of the oligonucleotides per 1.6 μL total volume of the thiol-maleimide PEG hydrogel in about 1 minute to about 35 minutes during rehydration. In some embodiments of the provided methods, a time required for a quantity to release half (t_{1/2}) of the oligonucleotides from the dried oligonucleotide-loaded thiol-maleimide PEG hydrogel-based matrix is from about 1 minute to 6 minutes during rehydration. In an embodiment, the 100 micron-flakes of the dried oligonucleotide-loaded thiol-maleimide PEG hydrogel-based matrix release the oligonucleotides in a near instantaneous release of less than one minute during rehydration. In a particular embodiments, from 550 μg to 997.50 μg oligonucleotides of a 950 μg to 1 mg loaded oligonucleotide mass is released during a rehydration period of from about 15 to 35 minutes. In some embodiments, the dried oligonucleotide-loaded thiol-maleimide PEG hydrogel-based matrix comprises 500 to 900 μg of oligonucleotides per 1.6 μL of total volume of the thiol-maleimide PEG hydrogel.

In one aspect, the present invention provides a method for formulating hydrogel matrix-encapsulated oligonucleotides in an amount that exceeds the oligonucleotides intrinsic solubility in water or aqueous media, the method comprising: (a) reacting a mixture of a maleimide functionalized polyethylene glycol (PEG-MAL) and a polyethylene glycol compound containing sulfhydryl groups (PEG-SH) together with an aqueous solution of oligonucleotides in a buffer having pH 4.0-4.8 to form a super-concentrated oligonucleotide-loaded hydrogel comprising a loading value of oligonucleotides of 400 μg per 1.6 μL total volume of the thiol-maleimide PEG hydrogel; and (b) casting the super-concentrated oligonucleotide-loaded hydrogel into a mold to create a uniform super-concentrated oligonucleotide-loaded thiol-maleimide PEG hydrogel-based matrix. In an embodiment, the method further comprises preparing the super-concentrated aqueous solution of oligonucleotides by heating an aqueous solution of oligonucleotides to 60° C. with simultaneous sonication. In some embodiments, the aqueous solution of oligonucleotides comprises 25-mer poly-dT. In certain embodiments, the method further comprises drying the uniform super-concentrated oligonucleotide-loaded hydrogel-based matrix at ambient temperature for 72 hours to form a dried super-concentrated oligonucleotide-loaded thiol-maleimide PEG hydrogel-based matrix comprising a 60% to 40% ratio of the oligonucleotide to the thiol-maleimide PEG. In an embodiment, the method further comprises slicing the dried super-concentrated oligonucleotide-loaded thiol-maleimide PEG hydrogel-based matrix into 100 micron-flakes. In some embodiments of the provided methods, density of the oligonucleotides is 1.4-1.7 g cm⁻³. In an embodiment, the super-concentrated oligonucleotide-loaded hydrogel comprises oligonucleotides, e.g., DNA, in a buffer having a pH of from 4.0 to 4.8, a polyethylene glycol compound containing sulfhydryl groups (PEG-SH) and a maleimide functionalized polyethylene glycol (PEG-MAL). In a particular embodiment of the super-concentrated oligonucleotide-loaded hydrogel, a ratio of oligonucleotides to PEG-SH to PEG-MAL is 7:1:2. In a specific embodiment, the super-concentrated oligonucleotide-loaded hydrogel solution comprises 7 μl oligonucleotides, e.g., DNA, in a buffer having a pH of from 4.0 to 4.8, 1 μl PEG-SH and 2 μl PEG-MA in a total volume of 10 In certain embodiments, the super-concentrated oligonucleotide-loaded hydrogel is miniaturized in a cylinder or pipette tip, each of which have dimensions of 1 mm diameter, 1-2 mm length and volume of from 0.8 to 1.6 μL. In a specific embodiment, 1.6 μL of the super-concentrated oligonucleotide-loaded hydrogel is cast in a pipette tip with a length of 2 mm (having an average diameter of approximately 1 mm) to form a super-concentrated oligonucleotide-loaded hydrogel pellet (also called a “hydrogel pellet” herein) having a cylindrical shape, and allowed to set for about 10 minutes, and then is removed from the pipette tip; oligonucleotide release is measured by immersion of the hydrogel pellet in 1 mL water with end-over-end mixing at room temperature. The DNA concentration in the supernatant and DNA release from the hydrogel matrix is monitored with A₂₆₀. (kinetic monitoring). As used herein, “super-concentrated” and “highly concentrated” are used interchangeably to mean an oligonucleotide load of about greater than 40% w/w to greater than 95% w/w oligonucleotide in the dried thiol-maleimide PEG hydrogel-based matrix. In an embodiment, a hydrogel of the present invention can be made about 6.7 times larger (10.7 μL) and dried to yield an oligonucleotide-load hydrogel polymer network of the present invention of the same mass and a slightly smaller volume (than previously made hydrogels comprising 85% water and 9% DNA) comprising an oligonucleotide, e.g., DNA, density of 1.4-1.7 g cm⁻³ and a PEG density of 1.1 g cm⁻³ in dry hydrogel. The methods of the present invention thus increase the loaded mass (concentration) of oligonucleotides in the dried super-concentrated oligonucleotide-loaded thiol-maleimide PEG hydrogel-based matrix (e.g., the hydrogel pellet) compared to prior hydrogels not made according to the methods described herein.

In various embodiments, oligonucleotides, e.g., DNA, is rapidly released from the hydrogel matrix, wherein the time required for a quantity to release half (t_{1/2}) of the oligonucleotides from the dried super-concentrated oligonucleotide-loaded PEG hydrogel-based matrix in the water or aqueous media is about 1 minute. Quantitative release is measured within 20 minutes. In particular embodiments, released mass correlates well with the loaded mass of oligonucleotides. Nucleic acid absorbance spectra have a peak at 260 mm in the UV range. This A₂₆₀ value is directly proportional to the nucleic acid concentration.

In particular embodiments of the provided methods, the dried super-concentrated oligonucleotide-loaded thiol-maleimide PEG hydrogel-based matrix releases from 0.025 mg to 1 mg of the oligonucleotides per 1.6 μL total volume of the thiol-maleimide PEG hydrogel in 0 to 15 minutes during a rehydration period. In some embodiments, a time required for a quantity to release half (t_{1/2}) of the oligonucleotides from the dried super-concentrated oligonucleotide-loaded thiol-maleimide PEG hydrogel-based matrix in the water or aqueous media is from about 1 minute to 6 minutes during a rehydration period. In an embodiment of the provided methods, the 100 micron-flakes of the dried super-concentrated oligonucleotide-loaded thiol-maleimide PEG hydrogel-based matrix in the water or aqueous media release the oligonucleotides in a near instantaneous release of less than one minute during a rehydration period. In a particular embodiment of the provided methods, 550 μg oligonucleotides of a 950 μg loaded oligonucleotide mass is released during the rehydration period. In some embodiments of the provided methods, the super-concentrated oligonucleotide-loaded hydrogel is cast into a mold having length and diameter dimensions on a micron scale up to a 2 mm length×10 mm diameter. In certain embodiments, the super-concentrated oligonucleotide-loaded hydrogel is cast into a conventional pipette tip to form a microcylinder of 2 mm length×1 to 2 mm diameter dimensions. In a particular embodiment of the provided methods, the super-concentrated oligonucleotide-loaded microcylinder (e.g., the hydrogel pellet) releases 110 μg of the oligonucleotides of the 400 μg loaded oligonucleotides from the dried 1.6 μL hydrogel microcylinder within 1 minute post-immersion/rehydration in water, aqueous media or body fluids or organs.

In another aspect, the present invention provides a formulation of hydrogel matrix-encapsulated super-concentrated oligonucleotides comprising a reaction mixture of a maleimide functionalized polyethylene glycol (PEG-MAL) and a polyethylene glycol compound containing sulfhydryl groups (PEG-SH) together with an aqueous solution of oligonucleotides in a buffer having pH 4.0-4.8, wherein the super-concentrated oligonucleotides comprise a loading value of oligonucleotides of 400 μg per 1.6 μL total volume of the thiol-maleimide PEG hydrogel.

In an embodiment of the formulation, the super-concentrated aqueous solution of oligonucleotides comprises 25-mer poly-dT. In some embodiments of the formulation, the super-concentrated aqueous solution of oligonucleotides is cast is a mold. In a particular embodiment of the provided formulation, the hydrogel matrix-encapsulated super-concentrated oligonucleotides is a dried formulation comprising a 60% to 40% ratio of the oligonucleotide to the thiol-maleimide PEG. In another embodiment, the dried rapid-release high concentration oligonucleotide-loaded thiol-maleimide PEG hydrogel-based matrix comprises an oligonucleotide load of about greater than 40% w/w to greater than 95% w/w oligonucleotide in the dried thiol-maleimide PEG hydrogel-based matrix. In some embodiments of the dried formulation, the dried super-concentrated aqueous solution of oligonucleotides is sliced into 100 micron-flakes. In certain embodiments of the dried formulation, density of the oligonucleotides is 1.4-1.7 g cm³. In a particular embodiment of the provided dried formulation, the dried formulation releases from 0.025 mg to 1 mg of the oligonucleotides per 1.6 μL total volume of the thiol-maleimide PEG hydrogel in 0 to 15 minutes during a rehydration period. In an embodiment of the dried formulation, a time required for a quantity to release half (t_{1/2}) of the oligonucleotides from the dried super-concentrated oligonucleotide-loaded thiol-maleimide PEG hydrogel-based matrix of from about 1 minute to 6 minutes. In some embodiments of the dried formulation, the 100 micron-flakes of the dried super-concentrated oligonucleotide-loaded thiol-maleimide PEG hydrogel-based matrix release the oligonucleotides in a near instantaneous release of less than one minute during a rehydration period. In a particular embodiment of the dried formulation, the 100 micron-flakes of the dried super-concentrated oligonucleotide-loaded thiol-maleimide PEG hydrogel-based matrix release 550 μg oligonucleotides of a 950 μg loaded oligonucleotide mass during the rehydration period. In some embodiments of the formulation, the super-concentrated oligonucleotide-loaded hydrogel is cast into a mold having length and diameter dimensions on a micron scale up to a 2 mm length×10 mm diameter. In an embodiment, the super-concentrated oligonucleotide-loaded hydrogel is cast into a conventional pipette tip to form a microcylinder of 2 mm length×1 to 2 mm diameter dimensions. In another particular embodiment of the dried formulation, the microcylinder (i.e., the hydrogel pellet) releases 110 μg of the 400 μg loaded oligonucleotides from the dried 1.6 μL hydrogel microcylinder within 1 minute post-immersion/rehydration in water, aqueous media, or body fluids or organs.

In one aspect, the present invention provides a method for systemic and local micro-delivery of therapeutic oligonucleotides, comprising administering to a subject in need thereof the 100 micron-flakes of the dried super-concentrated oligonucleotide-loaded PEG hydrogel-based matrix (e.g., a thiol-maleimide PEG hydrogel-based matrix) prepared by the methods described herein. In an embodiment of the provided methods, the 100 micron-flakes of the dried super-concentrated oligonucleotide-loaded PEG hydrogel-based matrix are administered to the central nervous system by implantation of the 100 micron-flakes to an anatomical locus of the subject for local micro-delivery of the therapeutic oligonucleotides. The particular embodiments, the dried super-concentrated oligonucleotide-loaded PEG hydrogel-based matrix comprises the therapeutic oligonucleotides in a therapeutically effective amount. In an embodiment, the 100 micron-flakes of the dried super-concentrated oligonucleotide-loaded PEG hydrogel-based matrix are administered as flattened and cut flakes. In an embodiment, dried super-concentrated oligonucleotide-loaded PEG hydrogel-based matrix is administered in a mold-cast miniaturized shape, e.g., a cylindrical shape, after casting in a pipette tip having a length of 2 mm and an average diameter of about 1 mm, described herein. In various embodiments, the dried super-concentrated oligonucleotide-loaded PEG hydrogel-based matrix is administered as a flat miniaturized hydrogel matrix. Administration of the mold-cast miniaturized dried super-concentrated oligonucleotide-loaded PEG hydrogel-based matrix and/or the flattened and cut 100 micron flakes is performed by systemic or local routes; in particular embodiments, the administration thereof is by local micro-delivery. In an embodiment, local micro-delivery to the central nervous system, bypasses the blood brain barrier (BBB). In a specific embodiment the mold-cast miniaturized dried super-concentrated oligonucleotide-loaded PEG hydrogel-based matrix and/or the flattened and cut 100 micron flakes are micro-delivered via implantation to the anatomical locus, which may be an organ or system in need of therapy of the subject. In some embodiments of the provided methods, the anatomical locus is a brain or a spine. In certain embodiments of the provided methods, the 100 micron-flakes are administered systemically by an enteral or parenteral administration. In some embodiments, the miniaturized dried super-concentrated oligonucleotide-loaded PEG hydrogel-based matrix and/or the flattened and cut 100 micron flakes are administered in mold-cast miniaturized form without a carrier. In certain embodiments, the miniaturized dried super-concentrated oligonucleotide-loaded PEG hydrogel-based matrix and/or the flattened and cut 100 micron flakes are administered in mold-cast miniaturized form with a carrier, e.g., for systemic delivery. In a particular embodiment of the provided methods, the dried super-concentrated oligonucleotide-loaded PEG hydrogel-based matrix is cast into a mold having length and diameter dimensions on a micron scale up to a 2 mm length×10 mm diameter. In some embodiments of the provided methods, the dried super-concentrated oligonucleotide-loaded PEG hydrogel-based matrix is cast into a conventional pipette tip to form a microcylinder of 2 mm length×1 to 2 mm diameter dimensions. In a particular embodiment of the provided methods, the super-concentrated oligonucleotide-loaded microcylinder (e.g., the hydrogel pellet) releases 110 μg of the 400 μg loaded oligonucleotides from the dried 1.6 μL hydrogel microcylinder within 1 minute post-immersion/rehydration in water, aqueous media or body fluids or organs. In an embodiment of the provided methods, the dried super-concentrated aqueous solution of oligonucleotides comprises 25-mer poly-dT. In certain embodiments, the 100 micron-flakes have a density of the oligonucleotides of from 1.4 to 1.7 g cm³. In particular embodiments, the dried super-concentrated oligonucleotide-loaded PEG hydrogel-based matrix comprises a 60% to 40% ratio of the oligonucleotide to the PEG. In some embodiments of the provided methods, the dried super-concentrated oligonucleotide-loaded PEG hydrogel-based matrix releases from 0.025 mg to 1 mg of the oligonucleotides per 1.6 μL total volume of the PEG hydrogel in 0 to 15 minutes during a rehydration period. In some embodiments of the provided methods, a time required for a quantity to release half (t_{1/2}) of the oligonucleotides from the dried super-concentrated oligonucleotide-loaded PEG hydrogel-based matrix in the water or aqueous media is from about 1 minute to 6 minutes during a rehydration period. In some embodiments, the 100 micron-flakes of the dried super-concentrated saturated oligonucleotide-loaded PEG hydrogel-based matrix in the water or aqueous media release the oligonucleotides in a near instantaneous release of less than one minute during a rehydration period. In a particular embodiment of the provided methods, 550 μg oligonucleotides of a 950 μg loaded oligonucleotide mass is released during the rehydration period. In a particular embodiment of the provided therapeutic methods, the dried super-concentrated oligonucleotide-loaded PEG hydrogel-based matrix comprises 400 μg of oligonucleotides per 1.6 μL total volume of the PEG hydrogel.

Therapeutically effective doses of the dried super-concentrated oligonucleotide-loaded PEG hydrogel-based matrix of the present invention or pharmaceutical compositions comprising the oligonucleotide-loaded PEG hydrogel-based matrix for treatment of conditions or diseases vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The dried super-concentrated oligonucleotide-loaded PEG hydrogel-based matrix of the invention or pharmaceutical compositions comprising the dried super-concentrated oligonucleotide-loaded PEG hydrogel-based matrix, wherein the active components are the oligonucleotides, thus may include a “therapeutically effective amount.” A “therapeutically effective amount” refers to an amount effective, at dosages and for periods of time necessary, to achieve the desired therapeutic result. A therapeutically effective amount of a molecule, in particular embodiments, the oligonucleotides administered for therapy of the subject, may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass a total amount of each active component, i.e., the oligonucleotides in the dried super-concentrated oligonucleotide-loaded PEG hydrogel-based matrix of the invention or pharmaceutical compositions comprising the dried super-concentrated oligonucleotide-loaded PEG hydrogel-based matrix or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. In some embodiments of the present invention, the active ingredient(s) is the super-concentrated oligonucleotide(s) loaded into the thiol-maleimide PEG hydrogel-based matrix according to the methods described herein. In a particular embodiment, the dried super-concentrated oligonucleotide-loaded PEG hydrogel-based matrix comprises a 60% to 40% ratio of the oligonucleotides to the PEG. In an embodiment, a density of the oligonucleotides in the PEG hydrogel-based matrix is 1.4-1.7 g cm⁻³. In certain embodiments, the super-concentrated oligonucleotide-loaded hydrogel comprises a ratio of oligonucleotides to PEG-SH to PEG-MAL of 7:1:2. In some embodiments, the super-concentrated oligonucleotide-loaded hydrogel solution, i.e., prior to drying, comprises 7 μl oligonucleotides, e.g., DNA, in a buffer having a pH of from 4.0 to 4.8, 1 μl PEG-SH and 2 μl PEG-MA in a total volume of 10 μl. In a particular embodiment, the super-concentrated oligonucleotide-loaded hydrogel comprises 1.6 μL of the super-concentrated oligonucleotide-loaded hydrogel cast in a mold, such as a pipette tip having a length of 2 mm and an average diameter of about 1 mm; such a mold-cast super-concentrated oligonucleotide-loaded hydrogel as used herein is called a “miniaturized super-concentrated oligonucleotide-loaded hydrogel” or a “miniaturized super-concentrated oligonucleotide-loaded thiol-maleimide PEG hydrogel-based matrix”. In an embodiment of the super-concentrated oligonucleotide-loaded hydrogel, the time required for a quantity to release half (t_{1/2}) of the oligonucleotides from the dried super-concentrated oligonucleotide-loaded thiol-maleimide PEG hydrogel-based matrix in the water or aqueous media is from about 1 minute to 6 minutes during a rehydration period. In an embodiment, the super-concentrated oligonucleotide-loaded hydrogel comprises 0.52 mg DNA loaded therein and prepared according to the methods described herein and provides a 78% release of the oligonucleotide upon rehydration in water or an aqueous media during a rehydration period, e.g., after administration to the subject, of 0-15 minutes with estimated t_{1/2} of 6 mins making it suitable for enhanced (i.e., increased) delivery of amounts of up to 1 mg of the oligonucleotides per a 1.6 μL matrix pellet. In a particular embodiment of the dried super-concentrated oligonucleotide-loaded hydrogel formulation, the super-concentrated oligonucleotide-loaded microcylinder (e.g., the hydrogel pellet) releases 110 μg of the 400 μg loaded oligonucleotides from the dried 1.6 μL hydrogel microcylinder within 1 minute post-immersion/rehydration in the in water or aqueous media or body fluids or organs.

When applied to a combination, the term active agent refers to combined amounts of the active ingredients that result in the therapeutic effect, whether administered in combination, serially or simultaneously.

The following examples are presented in order to more fully illustrate certain embodiments of the invention. They should in no way be construed, however, as limiting the broad scope of the invention.

### EXAMPLES

### Example 1

### Hydrogel (Matrix) Formulation

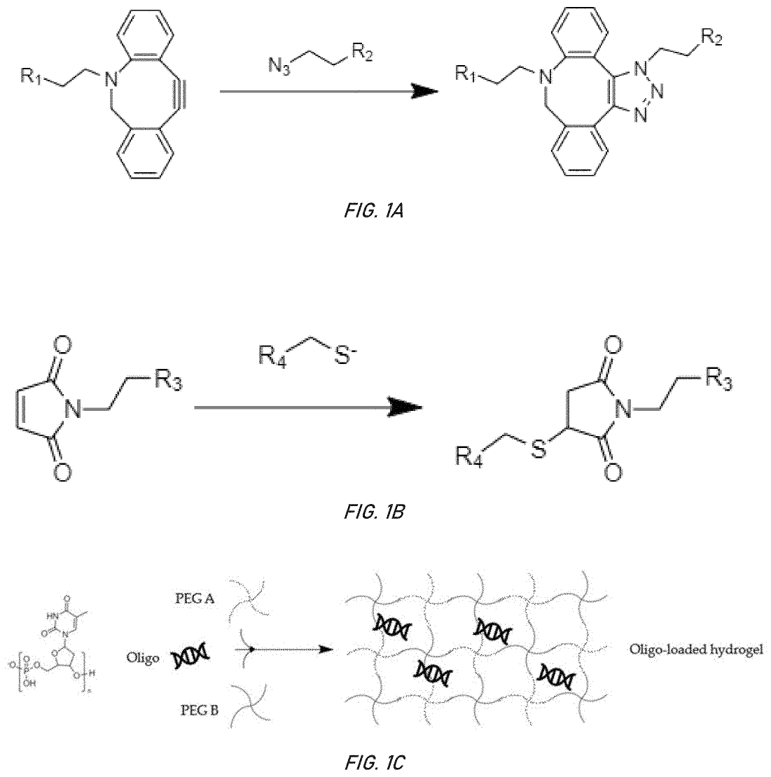

In an effort to identify an effective hydrogel matrix to encapsulate oligonucleotides in high concentration, multiple validated chemistries were evaluated; two specific examples, strain promoted azide alkyne cycloaddition and thiol-maleimide (thiol-Michael addition) are summarized in FIGS. 1A-1B. Both reactions were orthogonal to DNA functional groups. (FIG. 1C)

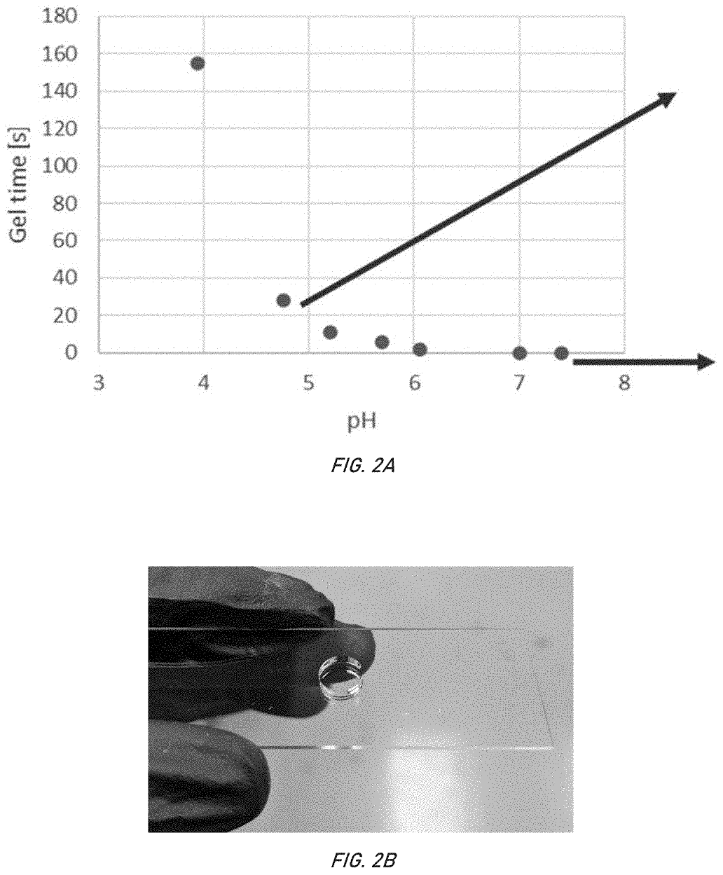

Following a series of experiments to identify most an expeditious and practical approach towards the aforementioned goal, efforts were focused on the thiol-Michael chemistry. Specific reasons included: (i) robust and controlled formation of the targeted gel in (ii) predictable fashion (time, viscosity) using (iii) versatile reagents, (iv) regimented pH range that allows for practical generation of the gel that could be (v) easily cast into a mold to create the desired hydrogel network suitable for the oligonucleotide encapsulation. FIG. 1B shows the thiol-Michael reaction is dependent on the thiolate anion (k˜10⁶ M¹ s⁻¹). Thiol-Michael hydrogels formed in a pH dependent reaction rate (FIG. 2A). The buffer contained 0.1 M histidine HCl (various pH). The gel point was measured as the time at which pipetting becomes impractical. FIG. 2B shows that at pH 4.7 a gel forms in 28 seconds. This was enough time to mix the hydrogel and oligonucleotide components thoroughly and cast the mixture into a mold to create a uniform hydrogel network. At pH 7.4, a gel forms instantaneously upon mixing and the hydrogel-oligonucleotide mixture was stuck in the pipette tip, as shown in FIG. 2C.

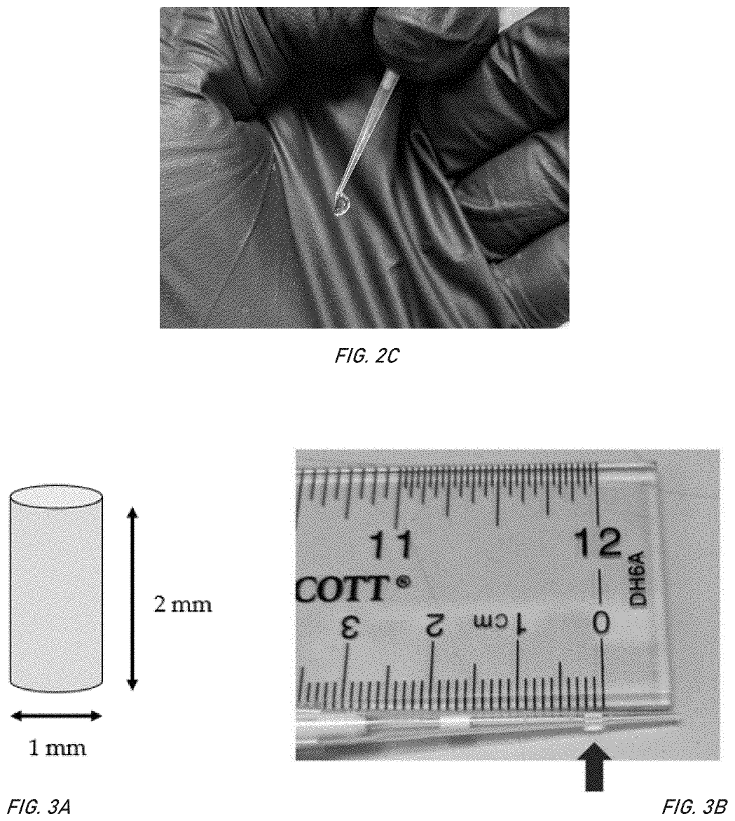

As illustrated in FIGS. 3A-3B, the resulting thiol-Michael hydrogels allowed for a facile shaping into both mini and micro shapes determined by the specific mold. In a representative example, an estimated 1.6 μL of the aforementioned hydrogel-oligonucleotide mixture was successfully cast using a conventional pipette tip to result in a microcylinder featuring 2 mm (length)×1 mm (diameter) dimensions. Importantly, size/dimension(s) of the desired matrix suitable for formulation could be reduced further to a micron scale or enlarged to 10 mm size depending on the specific therapeutic indication (safe dimensions, biocompatibility, desired therapeutic concentration and/or release pattern of the encapsulated therapeutics).

### Example 2

### Hydrogel Miniaturization

Hydrogel miniaturization was performed with dimensional constraints of 1 mm diameter, 1-2 mm length, and a volume of 0.8 to 1.6 μL of the hydrogel-oligonucleotide mixture (FIG. 3A); 1.6 μL of the hydrogel-oligonucleotide mixture was cast in a pipette tip (FIG. 3C) having a length of 2 mm and an average diameter of about 1 mm. FIGS. 3D-3E show the cast miniaturized hydrogel-oligonucleotide.

### Example 3

### Release Kinetics of Oligonucleotide from Hydrogel

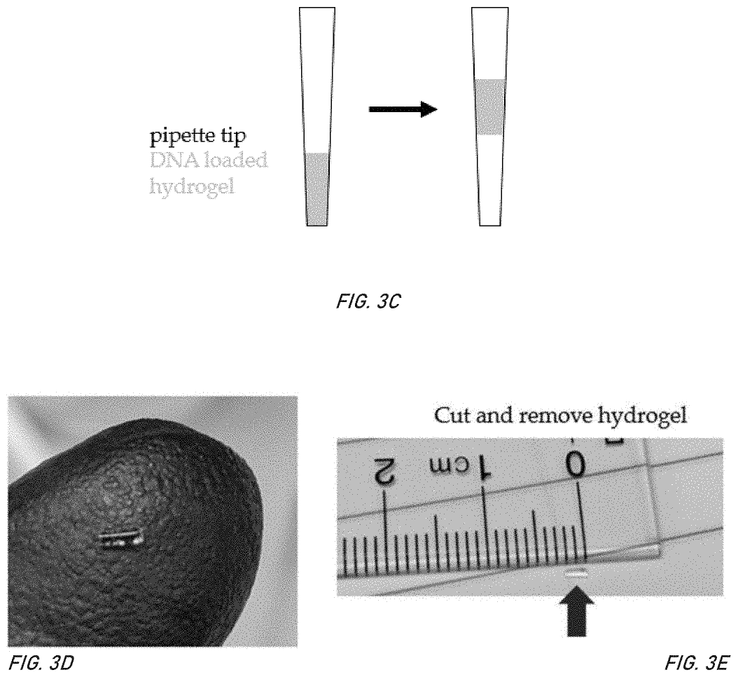

Following the above-described hydrogel formation studies, the encapsulation and release kinetics of a model antisense oligonucleotide (ASO) were studied, namely a poly-dT (25-mer). The specific protocol for the procedure is summarized in the FIG. 4. The release kinetics were monitored in water via absorption at 260 nm wavelength (A₂₆₀). Table 1 shows the components of the hydrogel-oligonucleotide formulation. 1.6 μl of the hydrogel-oligonucleotide mixture was cast in a pipette tip, allowed to set for 10 minutes, then removed and immersed in 1 mL water with end-over-end mixing at room temperature. The DNA concentration in the supernatant was monitored with A₂₆₀.

TABLE 1
Hydrogel Formulation Precursor

| | Volume |
|---|---|
| Component | (μL) |
| DNA in buffer pH 4.0-4.8 | 7 |
| PEG-SH | 1 |
| PEG-MAL | 2 |
| Total | 10 |

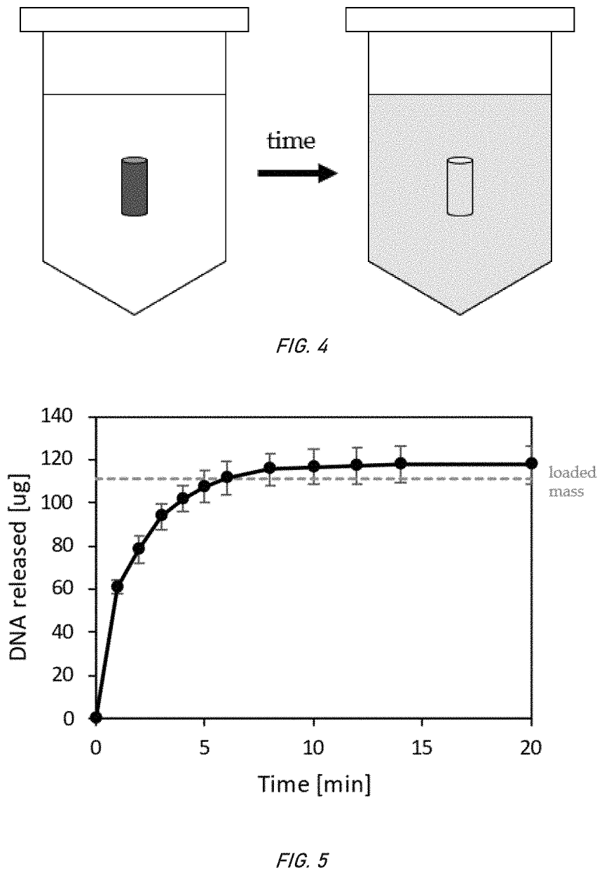

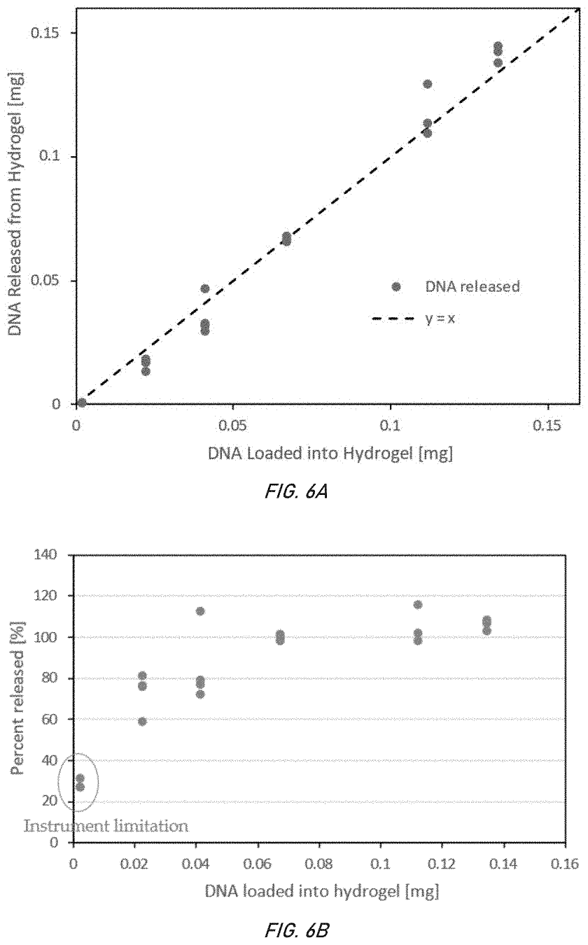

The resulting ASO release kinetics is shown in FIG. 5. The thiol-Michael gel-encapsulated poly-dT was released from the hydrogel rapidly within 1 minute post-immersion in water to afford quantitative yield of the oligonucleotide as measured by the absorption. Notably, the amount of the oligonucleotide encapsulated and released by the aforementioned 2 mm×1 mm hydrogel (1.6 μL) pellet was estimated to be about 110 μg as evidenced by the plot in FIG. 5. Quantitative release was measured within 20 minutes. The released oligonucleotide mass correlated well with the loaded oligonucleotide mass. (A_{260, min}=A_{260, 24 h}). FIGS. 6A-6B show that oligonucleotide mass determined release of the oligonucleotide after the DNA-loaded hydrogel is immersed in water.

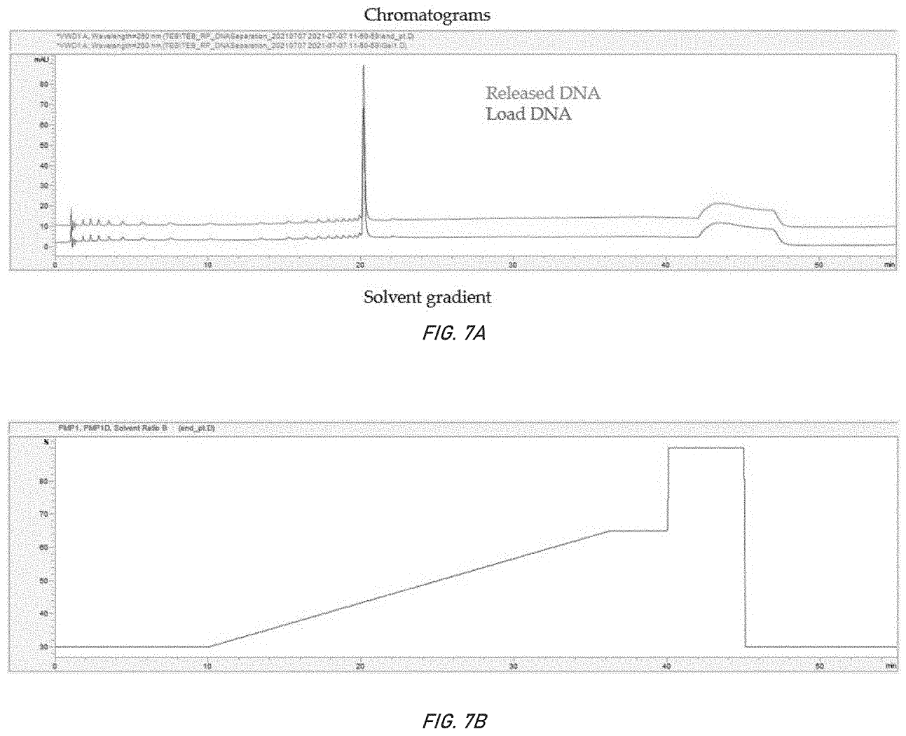

Both the identity as well as the integrity of the model oligonucleotide was confirmed by reverse-phase HPLC as shown in FIGS. 7A-7B to fully suggest that poly-dT could be reliably identified and quantified and that it is stable during the protocol including both encapsulation and the quantitative release. FIG. 7A shows that the loaded and released DNA chromatograms are identical. The DNA that reacted with PEG is not likely to be released because the DNA is covalently attached to the hydrogel network. FIG. 7B shows solvent gradient HPLC of the hydrogel-encapsulated DNA. The column was Waters XBridge™ C18 3.5 μm. Solvent A was 0.1 M triethylammonium acetate (TEAA) in water; Solvent B was 0.1 M TEAA in 80/20% (H₂O/Acetonitrile). The solvent gradient HPLC was performed at a temperature of 60° C. and a flow rate of 1 mL/min.

### Example 4

### Super-Concentrated (High Concentration) Oligonucleotide Hydrogel Formulations

Next, an attempt was made to increase the concentration of the model oligonucleotide entrapped by the hydrogel via increasing its concentration in the parent solution. The rationale was to arrive at the matrix-based formulations of oligonucleotides that exhibit increased concentration of the targeted molecule to match therapeutic concentrations observed in the clinical setting. In a representative example, HTT-targeting antisense oligonucleotide Tominersen™ (Roche) is administered at a bolus intrathecal (IT) injection in the cerebrospinal fluid (CSF) at 120 mg total dose per treatment. Taking into consideration the total volume of CSF of about 150 mL, the desired intraventricular concentration of the oligonucleotide molecule is approximately 0.8 mg/mL.

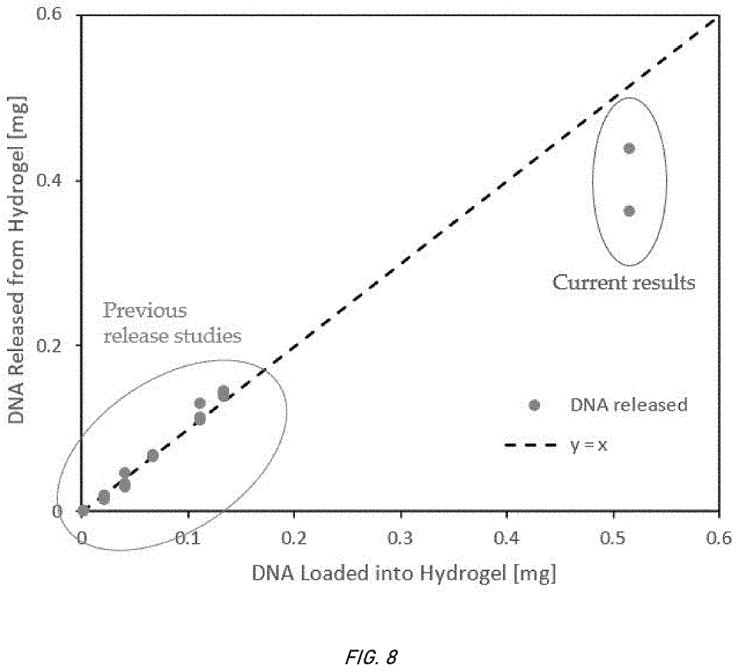

In an initial series of experiments, a series of (super)concentrated aqueous solutions of the model DNA (poly-dT, 25-mer) were prepared by heating the mixture to 60° C. with simultaneous sonication. In a representative example, a considerably higher entrapment of the targeted poly-dT by the thiol-Michael hydrogel was able to be achieved following the described mixing procedure to yield an estimated 400 microgram loading value per 1.6 μL total volume of the hydrogel (components in Table 1). As shown in FIG. 8, this result compares favorably with the earlier experiments following a more traditional innate solubility of the oligonucleotide. 1.6 μL total volume of the hydrogel formed as before (above); 0.52 ng DNA loaded into the hydrogel mixture released 78% of the DNA.

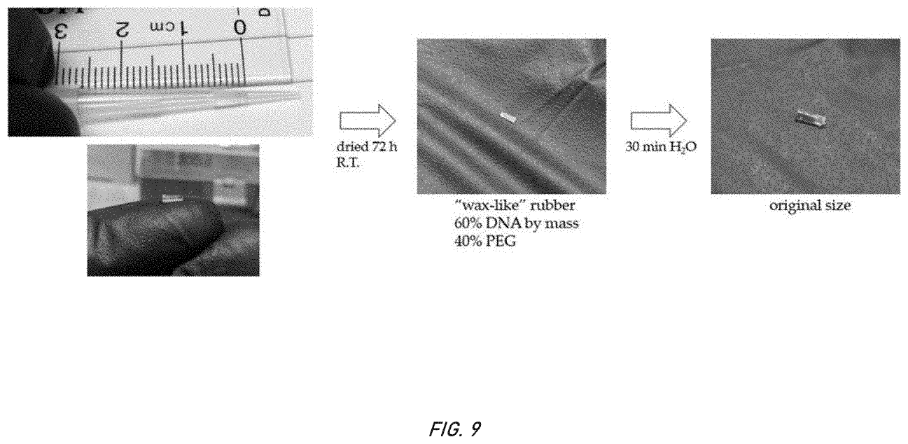

Next, an attempt to reduce the amount of the entrapped water in the thiol-Michael hydrogel-poly-dT matrix was made. The hydrogel matrix obtained in the previous experiments was dried for 72 hours at ambient temperature to result in an oligonucleotide-hydrogel complex featuring 60%/40% ratio of the oligonucleotide to PEG. Furthermore, the matrix swelled in water (30 minutes treatment) to release the targeted poly-dT in a highly-predictable fashion. Previously, the hydrogels contained 85% water by mass (6 wt % PEG and wt % DNA). The 72-hour drying shown in FIG. 9 prepared an oligonucleotide-hydrogel matrix that is 6.7 times larger (10.7 μL) than the previous oligonucleotide-hydrogel matrix comprising 6 wt % PEG and wt % DNA and yielded a DNA-loaded polymer hydrogel network of the same mass and a slightly smaller volume. The density of the oligonucleotides (DNA) was 1.4-1.7 g cm⁻³ and the PEG density was 1.1 g cm³.

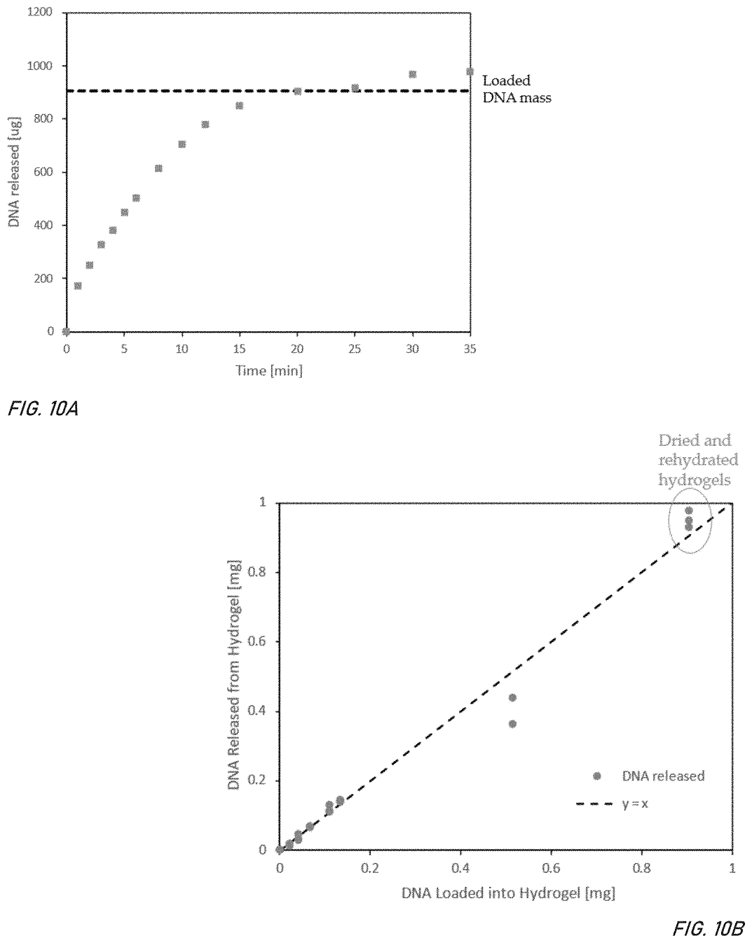

As evidenced by the release studies, including both loading and release kinetics, the described dried hydrogel accommodates increased quantities of oligonucleotides (FIGS. 10A-10B). The kinetics data suggest linear rates of release during 0-15 minute intervals with estimated t_{1/2} of 6 minutes, making it suitable for (i) enhanced delivery amounts of up to 1 mg of the oligonucleotide per 1.6 μL matrix pellet (versus 140 μg per same volume using ‘traditional’ technique) and (ii) predictable (fast) release kinetics. As shown in FIGS. 10A-10B, the dried hydrogels can be loaded with substantially more DNA and DNA release is delayed during rehydration. A delayed, nearly linear release phase during hydrogel re-hydration (˜10 minutes by eye) was demonstrated. The t_{1/2} was 6 minutes, compared to 1 minute for hydrated hydrogels. The 905 mg DNA loaded showed a 105% release.

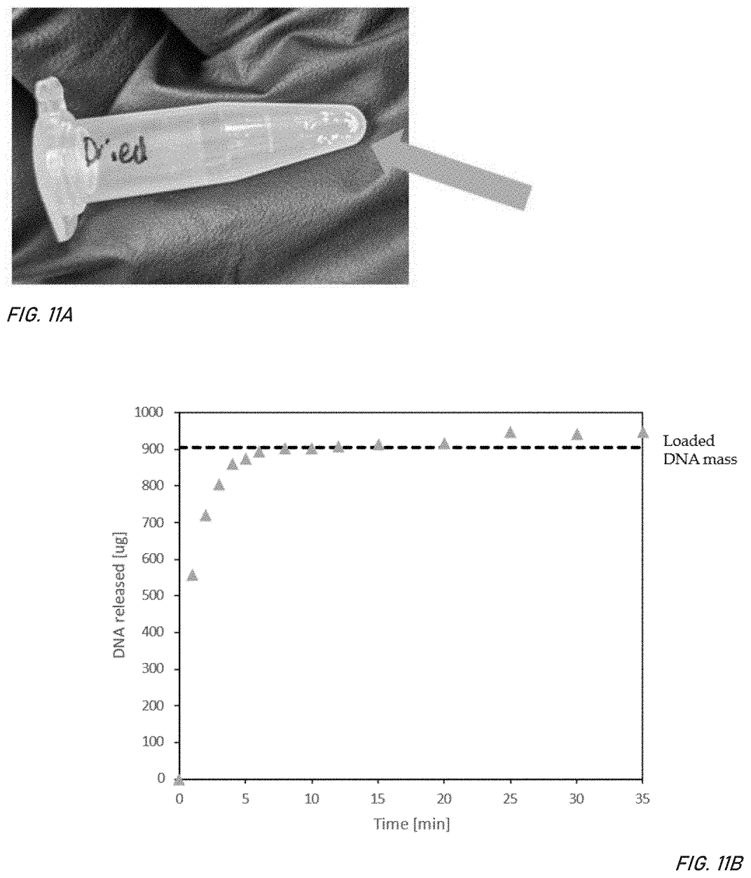

FIG. 11B shows the release kinetics of cut and flattened hydrogel flakes. The dried oligonucleotide-hydrogel formulation was flattened and cut into small flakes (FIG. 11A). The large surface area leads to rapid burst release of the DNA cargo from the hydrogel matrix (FIG. 11B).

Furthermore, the release kinetics of the described dried gels could be easily manipulated by processing them into regimented particles. In a representative example, a dried super-concentrated thiol-Michael-poly-dT gel was further sliced into ˜100 micron flakes, followed by kinetics studies to result in an almost instant (“burst”) release of the oligonucleotide as described below.

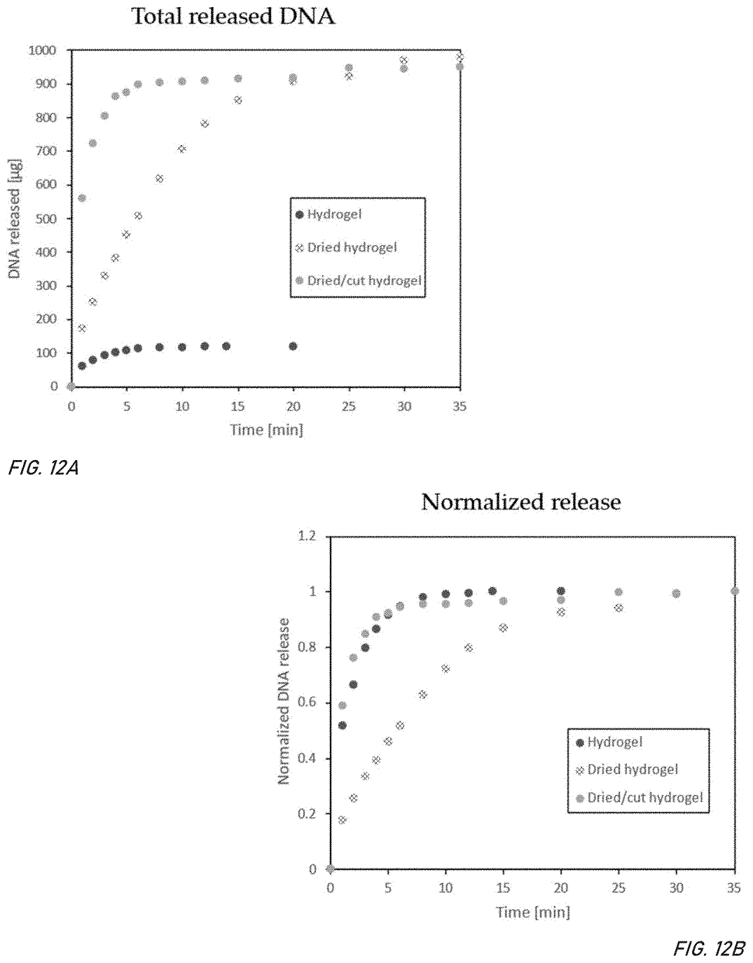

A comparison between the described protocols, including “traditional” solubility-limiting immobilization of oligonucleotides (black dots “hydrogel”) and the present approach (checkered dots: “dried” hydrogel, grey dots: dried/cut hydrogel) (FIGS. 12A-12B) suggests that the herein described novel procedure allows for (i) considerable enhancement of loading (almost 10-fold greater than “traditional” solubility-limiting immobilization of oligonucleotides) and (ii) regimented release kinetics ranging between almost instantaneous “burst” release for the dried/cut hydrogels and delayed release options. The delayed release option can be further modulated to achieve predictable minutes-to-days/weeks release of the therapeutic agent, e.g., oligonucleotides. Specifically, altering the (i) nature of the matrix, (ii) hydrogel dehydration protocol, (iii) further layering and/or encapsulation of the hydrogel-oligonucleotide complex, (iv) covalent, Van der Waals or ionic microenvironment within the hydrogel, and (v) modifying the actual therapeutic payload are expected to provide opportunities for further tuning of the herein described approach.

### Example 5

### Large Oligonucleotide Hydrogel Formulation

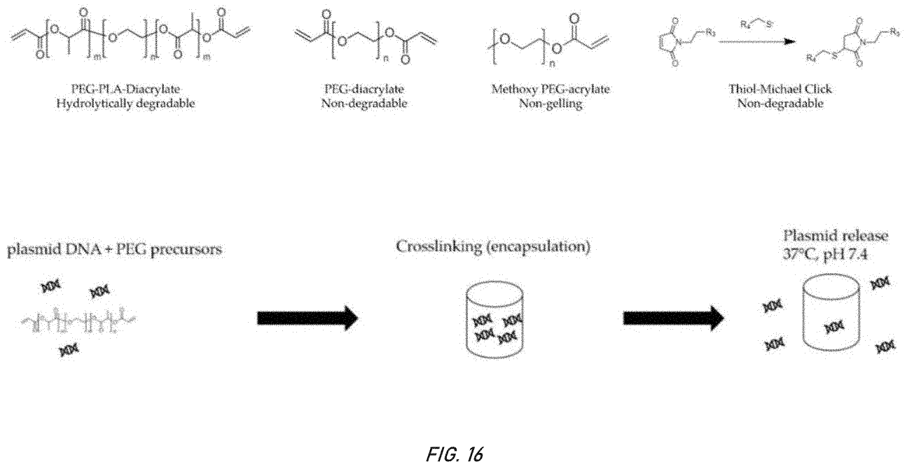

For a next series of experiments, attention was turned to larger biomolecules (than the relatively small-sized agents (<35-50 kD) described in Examples 1-4), namely DNA plasmids. A GFP plasmid (˜5.4 kbp) was selected as a model. Accordingly, a series of specific cross-linking agents/conditions, as well as the hydrogel-released conditions, were selected and validated to match the task in hand. Several representative monomers-gels and polymerization conditions evaluated are summarized in FIG. 16 and in Table 2 below:

TABLE 2
Large-Biomolecule Hydrogel Formulation Conditions

| Gel Type | Polymerization conditions |
|---|---|
| PEG-PLA-DA | 385 nm light; 5 minutes, 25 mW cm⁻², |
| (hydrolytically | 0.2% photoinitiator |
| degradable) | |
| PEG-DA | 385 nm light; 5 minutes, 25 mW cm⁻², |
| (non-degradable) | 0.2% photoinitiator |
| mPEG-A | 385 nm light; 5 minutes, 25 mW cm⁻², |
| (non-gelling control) | 0.2% photoinitiator |
| Thiol-Michael Click | 10 minutes pH 4.0 |
| (non-degradable) | |

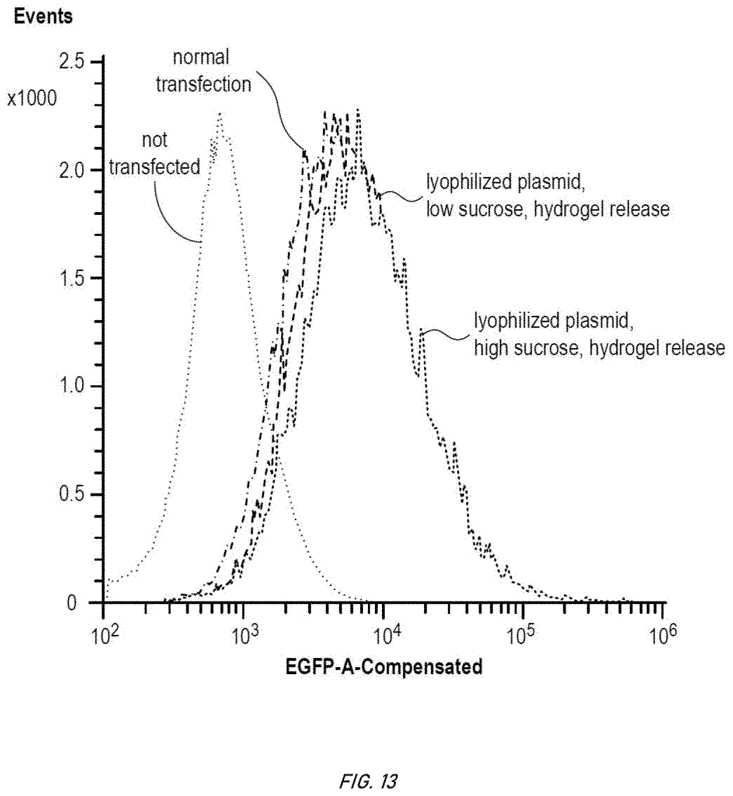

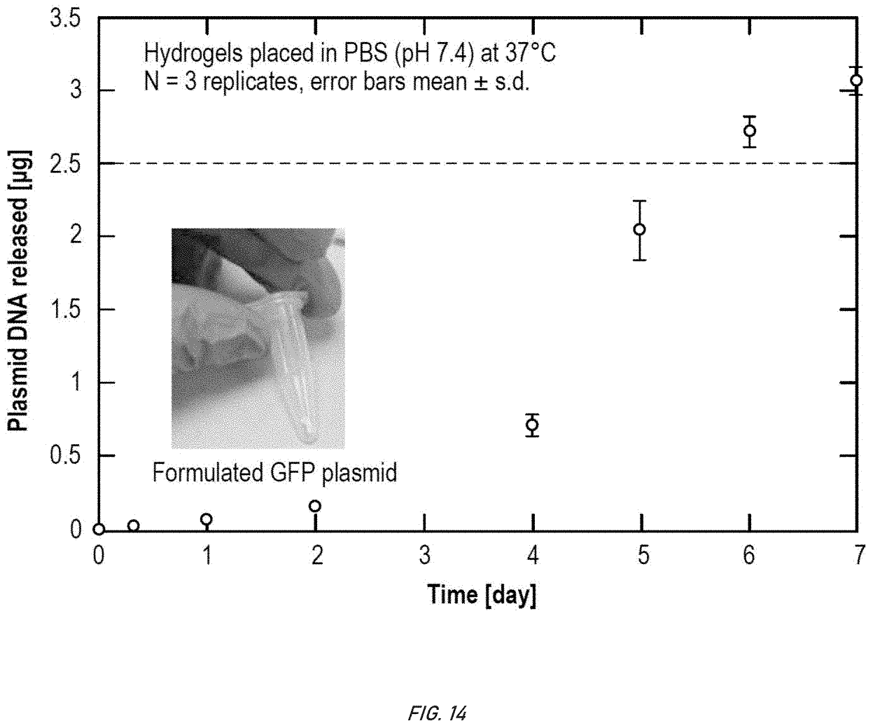

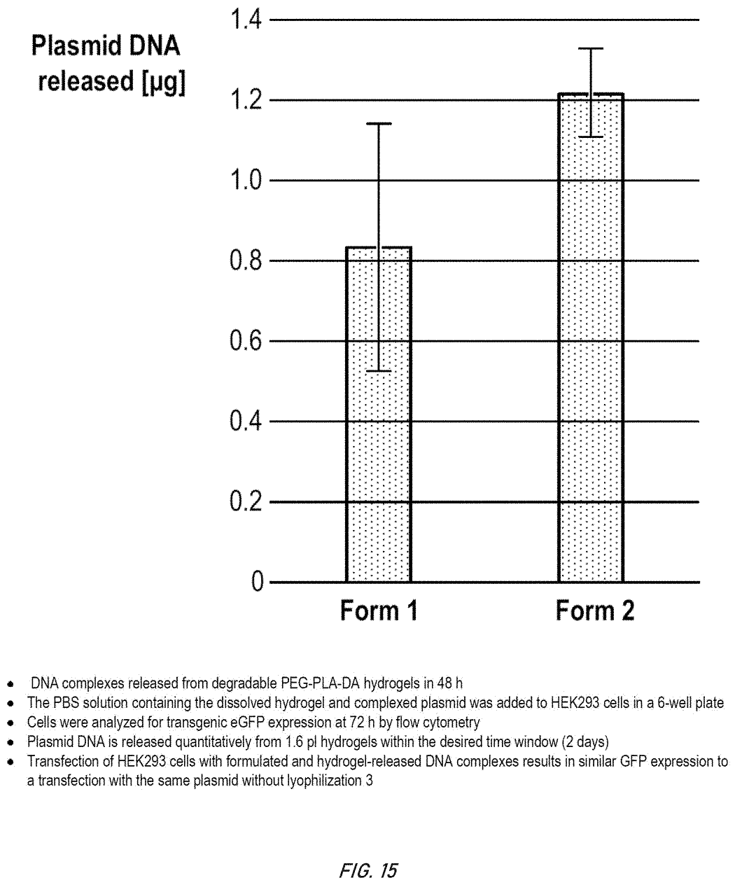

Following a series of combinatorial steps, an optimized hydrogel chemistry that accommodated the GFP plasmid was arrived at. Specific additives, including sucrose, aimed at enhancing both hydrolytic stability of the plasmid (FIGS. 13 and 15), as well as it is competence, dramatically enhanced the release of the plasmid and its transfection capacity. In FIG. 15, Form 1 (labeled as “low sucrose” in FIG. 13) has a sucrose to DNA ratio of 160:1 (by mass), while Form 2 (labeled as “high sucrose” in FIG. 13) has a sucrose to DNA ratio of 500:1 (by mass). Specifically, a robust release of the GFP plasmid at a theoretical loading level (˜2.75 μg per Bionaut™ pellet) (FIG. 14) was confirmed by measuring the expression levels of GFP protein in HEK293 cells and comparing it with a non-formulated control plasmid (FIG. 13).

In this Example, the optimized large-biomolecule hydrogel formulation for the stabilized plasmids allows for 5×to 10×enhancement of loading levels for such agents as compared to standard deliveries.

Of note, the herein described approach to formulating oligonucleotides is suitable for delivering a therapeutically relevant concentration/dose (i.e., therapeutically effective amount) of the agent (oligonucleotides) following local delivery, including implantation and/or any alternative localized delivery method, as represented by the Bionaut™ microrobot-mediated platform.

Any patent, patent application publication, or scientific publication, cited herein, is incorporated by reference herein in its entirety.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A dried rapid-release oligonucleotide-loaded polyethylene glycol (PEG) hydrogel-based matrix for delivery of a high concentration of oligonucleotides, the oligonucleotide-loaded PEG hydrogel-based matrix having a time required for a quantity to release half (t_{1/2}) of the oligonucleotides of from about 1 minute to about less than 30 minutes upon rehydration.

2. The dried rapid-release oligonucleotide-loaded PEG hydrogel-based matrix of claim 1, wherein the high concentration of oligonucleotides comprises greater than 40% w/w to 80% w/w or greater than 80% w/w oligonucleotide in the dried hydrogel-based matrix.

3-7. (canceled)

8. The dried rapid-release oligonucleotide-loaded PEG hydrogel-based matrix of claim 1 having a t_{1/2} of the oligonucleotides of from about 1 minute to about less than 20 minutes upon rehydration.

9. The dried rapid-release oligonucleotide-loaded PEG hydrogel-based matrix of claim 8, wherein rehydration is in water, aqueous media or body fluids selected from the group consisting of cerebrospinal fluid (CSF), blood, lymph, synovial fluid or aqueous humor.

10-16. (canceled)

17. The dried rapid-release oligonucleotide-loaded PEG hydrogel-based matrix of claim 1 having an average length of between 1 and 20 μm.

18-19. (canceled)

20. The dried rapid-release oligonucleotide-loaded PEG hydrogel-based matrix of claim 1, wherein the oligonucleotides comprise 25-mer poly-dT(s).

21. The dried rapid-release oligonucleotide-loaded PEG hydrogel-based matrix of claim 1, wherein density of the oligonucleotides is 1.4-1.7 g cm⁻³ and PEG density is 1.1 g⁻³ in a volume of 10.6 μL of the dried rapid-release oligonucleotide-loaded PEG hydrogel-based matrix.

22. The dried rapid-release oligonucleotide-loaded PEG hydrogel-based matrix of claim 1, which releases from 0.025 mg to 1 mg of the oligonucleotides per 1.6 μL total volume of the PEG hydrogel during a rehydration period of from less than one minute to 15 minutes.

23. The dried rapid-release oligonucleotide-loaded PEG hydrogel-based matrix of claim 1 comprising sliced and flattened 100 micron-long flakes.

24-26. (canceled)

27. The dried rapid-release oligonucleotide-loaded PEG hydrogel-based matrix of claim 1 which is loadable into a delivery device having a volume of 1 mm length by 1 mm width by 1 mm height.

28. The dried rapid-release oligonucleotide-loaded PEG hydrogel-based matrix of claim 1 comprising a reaction mixture of a maleimide functionalized polyethylene glycol (PEG-MAL) and a polyethylene glycol compound containing sulfhydryl groups (PEG-SH) together with an aqueous solution of oligonucleotides in a buffer having pH 4.0-4.8, wherein the oligonucleotides comprise a loading value of oligonucleotides of 500 to 900 μg per 1.6 μL total volume of thiol-maleimide PEG hydrogel-based matrix.

29. The dried rapid-release oligonucleotide-loaded PEG hydrogel-based matrix of claim 28 which is cast in a mold.

30-31. (canceled)

32. The dried rapid-release oligonucleotide-loaded PEG hydrogel-based matrix according to claim 1, wherein the PEG hydrogel-based matrix is a thiol-maleimide PEG hydrogel.

33. The dried rapid-release oligonucleotide-loaded PEG hydrogel-based matrix according to claim 1, wherein the PEG hydrogel-based matrix is a polyethylene glycol—polylactic acid—diacrylate (PEG-PLA-DA) hydrogel or a PEG-diacrylate (PEG-DA) hydrogel.

34. The dried rapid-release oligonucleotide-loaded PEG hydrogel-based matrix of claim 33, wherein the PEG-PLA-DA hydrogel or the PEG-DA hydrogel is photo-polymerized.

35.-36. (canceled)

37. A delivery device for delivery of a therapeutically effective amount of a high concentration of oligonucleotides to a specific tissue location in a subject, wherein the delivery device is loaded with the dried rapid-release oligonucleotide-loaded polyethylene glycol (PEG) hydrogel-based matrix according to claim 1.

38. The delivery device of claim 37 having a volume of 1 mm length by 1 mm width by 1 mm height.

39-60. (canceled)

61. A method for formulating dried rapid-release hydrogel matrix-encapsulated oligonucleotides in a high concentration that exceeds the oligonucleotides intrinsic solubility in water, aqueous media or body fluids, the method comprising:
a) reacting a mixture of a maleimide functionalized polyethylene glycol (PEG-MAL) and a polyethylene glycol compound containing sulfhydryl groups (PEG-SH) with a concentrated aqueous solution of oligonucleotides in a buffer having pH 4.0-4.8 to form an oligonucleotide-loaded hydrogel comprising a loading value of oligonucleotides of 500 to 900 μg per 1.6 μL total volume of the thiol-maleimide PEG hydrogel;
b) casting the oligonucleotide-loaded hydrogel into a mold to create a uniform oligonucleotide-loaded thiol-maleimide PEG hydrogel-based matrix; and
c) drying the uniform oligonucleotide-loaded hydrogel-based matrix at ambient temperature to form a dried oligonucleotide-loaded thiol-maleimide PEG hydrogel-based matrix comprising from greater than 40% w/w to 80% w/w or greater than 80% w/w oligonucleotide in the dried thiol-maleimide PEG hydrogel-based matrix.

62. The method of claim 61, further comprising preparing the concentrated aqueous solution of oligonucleotides by heating an aqueous solution of oligonucleotides to 60° C. with simultaneous sonication.

63. The method of claim 61, wherein the aqueous solution of oligonucleotides comprises 25-mer poly-dT.

64. The method of claim 61, wherein the drying of the uniform oligonucleotide-loaded hydrogel-based matrix occurs for 72 hours.

65. The method of claim 61, further comprising slicing the dried oligonucleotide-loaded thiol-maleimide PEG hydrogel-based matrix into 100 micron-flakes and flattening the flakes to between 1 micron and 100 microns in thickness.

66-70. (canceled)

71. The method of claim 61, wherein the mold has length and diameter dimensions on a micron scale up to a 2 mm length×10 mm diameter.

72. The method of claim 61, wherein the mold is a conventional pipette tip having length and diameter dimensions of 2 mm length×1 to 2 mm diameter and the casting forms a microcylinder.

73. (canceled)

74. A method for systemic or local micro-delivery of therapeutic oligonucleotides, the method comprising administering to a subject in need thereof 100 micron-flakes of a sliced and flattened dried rapid-release high concentration oligonucleotide-loaded polyethylene glycol (PEG) hydrogel-based matrix, the oligonucleotide-loaded PEG hydrogel-based matrix having a time required for a quantity to release half (tin) of the oligonucleotides of from about 1 minute to about less than 30 minutes upon rehydration.

75. The method of claim 74, wherein the 100 micron-flakes of the sliced and flattened dried rapid-release high concentration oligonucleotide-loaded PEG hydrogel-based matrix are administered to the central nervous system by implantation of the 100 micron-flakes to an anatomical locus of the subject for local micro-delivery of the therapeutic oligonucleotides.

76. The method of claim 75, wherein the anatomical locus is a brain or a spine.

77. The method of claim 75, wherein the 100 micron-flakes are administered systemically by an enteral or parenteral administration.

78. The method of claim 74, further comprising preparing the 100 micron-flakes, the method comprising:
a) casting into a mold having length and diameter dimensions of from a micron scale up to a 2 mm length×1 mm diameter a high concentration oligonucleotide-loaded thiol-maleimide PEG hydrogel comprising a loading value of oligonucleotides of 500 to 900 μg per 1.6 μL total volume of the thiol-maleimide PEG hydrogel to create a uniform oligonucleotide-loaded thiol-maleimide PEG hydrogel-based matrix;
b) drying the uniform oligonucleotide-loaded hydrogel-based matrix at ambient temperature to form a dried oligonucleotide-loaded thiol-maleimide PEG hydrogel-based matrix comprising from greater than 40% w/w to 80% w/w or greater than 80% w/w oligonucleotide in the dried thiol-maleimide PEG hydrogel-based matrix;
c) slicing the dried thiol-maleimide PEG hydrogel-based matrix into 100 micron-flakes; and
d) flattening the flakes to between 1 micron and 100 microns in thickness.

79. The method of claim 78, wherein the high concentration oligonucleotide-loaded thiol-maleimide PEG hydrogel is cast into a conventional pipette tip to form a microcylinder of 2 mm length×1 to 2 mm diameter dimensions.

80. The method of claim 79, wherein the microcylinder releases 110 μg of the oligonucleotides within 1 minute post-immersion/rehydration in the water or aqueous media or body fluids or organs.

81. The method of claim 78, further comprising preparing the high concentration oligonucleotides by heating an aqueous solution of oligonucleotides comprising 20 to 50% w/w oligonucleotide in the aaqueous solution to 60° C. with simultaneous sonication.

82. (canceled)

83. The method of claim 78, wherein the drying of the uniform oligonucleotide-loaded hydrogel-based matrix occurs for 72 hours.

84-91. (canceled)
